(19) United States
(12) Patent Application Publication
(10) Pub. No.: US 2024/0416152 A1
(43) Pub. Date: Dec. 19, 2024

(54) ACTIVATION OF LATE RESPONSE GENES USING NEUROMODULATION

(71) Applicant: GE Precision Healthcare LLC, Waukesha, WI (US)

(72) Inventors: Christopher Michael Puleo, Niskayuna, NY (US); Victoria Eugenia Cotero, Troy, NY (US)

(21) Appl. No.: 18/700,499

(22) PCT Filed: Oct. 12, 2022
(86) PCT No.: PCT/US2022/046440

§ 371 Date: Apr. 11, 2024

Related U.S. Application Data
(60) Provisional application No. 63/255,339, filed on Oct. 13, 2021.

(51) Int. Cl. A61N 7/02; A61N 7/00

(52) U.S. Cl. CPC A61N 7/02; A61N 2007/0026

(57) ABSTRACT

The present disclosure relates to the use of therapeutic ultrasound to non-invasively stimulate multiple peripheral nerve pathways that modulate energy homeostasis. An embodiment of the disclosed neuromodulation techniques includes neuromodulation techniques to treat a patient with a metabolic disorder. Certain embodiments of the disclosure are discussed in the context of blood glucose regulation.

### CROSS-REFERENCE TO RELATED APPLICATIONS

This application claims priority to and is a national stage filing of International Application No. PCT/US2022/046440, filed on Oct. 12, 2022, which claims priority to and the benefit of U.S. Provisional Application No. 63/255,339, entitled “ACTIVATION OF LATE RESPONSE GENES USING NEUROMODULATION” and filed Oct. 13, 2021, the disclosures of which are incorporated herein by reference for all purposes.

### BACKGROUND

The subject matter disclosed herein relates to neuromodulation and more specifically, to techniques by which the therapeutic use of ultrasound to non-invasively stimulate multiple peripheral nerve pathways may be used to modulate energy homeostasis. An embodiment of the disclosed neuromodulation techniques includes neuromodulation techniques to treat a patient with a metabolic disorder. Certain embodiments of the disclosure are discussed in the context of blood glucose regulation.

Type 2 diabetes (T2D) remains a common and costly disease worldwide. Despite significant pharmaceutical breakthroughs, T2D is still managed through frequent dosing of antidiabetic drugs that transiently ameliorate hyperglycemia, often requiring dose escalation and combination therapies with other drugs. This leaves patients vulnerable to co-morbidities, such as vascular and renal injury arising from a combination of diabetic nephropathy and drug-induced nephrotoxicity. Surgical approaches, such as bariatric surgery, remain the only clinical intervention shown to provide long-term remission from T2D. However, mechanisms underlying the long-term effects of the surgery remain controversial and have been shown to be secondary to weight loss.

Long-term remission from hyperglycemia has been demonstrated in T2D models using an invasive neuro-pharmaceutical approach. By way of example, a single treatment with recombinant fibroblast growth factor 1 (FGF1) has been shown to induce remission from hyperglycemia in diabetic mice and rats for weeks to months. However, this treatment has only been demonstrated to be effective when administered via invasive intracerebroventricular (i.e.v.) injection into the third ventricle, which incurs the risk associated with such an invasive, intracranial procedure.

### SUMMARY

The present disclosure relates the use of therapeutic ultrasound to non-invasively stimulate multiple peripheral nerve pathways known to modulate energy homeostasis. In certain embodiments a single ultrasound treatment session, in which both the hepatoportal and superior mesenteric plexuses are stimulated, was observed to induce sustained diabetes remission in animal models (e.g., the Zucker Diabetic Fatty (ZDF) and Diet-Induced Obesity (DIO) rodent models of type II diabetes (T2D)). The anti-diabetic effect of the ultrasound treatment is not secondary to weight loss and is achieved using ultrasound pulses that fall under current regulatory limits on ultrasound exposure. Correspondingly, it is believed that non-invasive ultrasound neuromodulation has the potential to modulate neurometabolic pathways and induce diabetes remission using a non-invasive and clinically relevant approach.

By way of further elaboration, and as discussed herein, stimulation of identified tissues (e.g., peripheral neural pathways) is performed for a sufficient length of time to induce a sustained change in membrane polarization in the plasma membrane or the nuclear membrane of certain cells. This change leads to sustained therapeutic changes, which in described examples is a change in the neural tissue of the hypothalamus.

By way of example, in certain implementations energy is directed to a target tissue (e.g., the hepatoportal plexus and/or superior mesenteric plexus) for a duration effective to activate late response genes in the hypothalamus. In particular, ion channel activation within the cells leads to sustained expression of immediate early genes for a duration effective to cause activation of the late response genes.

In one embodiment, a treatment method is provided. In accordance with this embodiment, a tissue of a subject is non-invasively stimulated for a duration that is effective to activate a late-response gene in the subject. The activation of the late-response gene causes expression of a gene-expression product that effects energy homeostasis.

In a further embodiment, a multi-site neuromodulation treatment method is provided. In accordance with this embodiment, non-invasive multi-site neuromodulation is applied to modulate more than one peripheral nerve pathway. The peripheral nerve pathways are in communication with each other or with an integrating neuron or cell in the central nervous system. The multi-site neuromodulation causes activation of a late-response gene that causes expression of a gene-expression product that effects energy homeostasis.

In an additional embodiment, a method for changing neural sensitivity is provided. In accordance with this method, one or more peripheral neural pathways are neuromodulated. The act of neuromodulating the tissue comprises activation of an ion channel selected from the TRP family of ion channels.

In a further embodiment, a system for treating a metabolic disorder in a subject is provided. In accordance with this embodiment, the system comprises an energy application device comprising at least one ultrasound transducer configured to non-invasively target a tissue of the subject and a pulse generator configured to be connected to the at least one ultrasound transducer to stimulate the tissue using the at least one ultrasound transducer for a duration that is effective to thereby activate late response genes in the subject and thereby treat or alleviate the metabolic disorder.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts the neuropeptide Y (NPY) neural pathway and the proopiomelanocortin (POMC) neural pathway;

FIG. 2A illustrates the anatomical location of the hepatoportal plexus;

FIG. 2B illustrates the anatomical location of the superior mesenteric plexus;

FIG. 3 illustrates that dual-site peripheral focused ultrasound stimulation (pFUS) of both the liver (i.e., hepatoportal plexus) and GI (i.e., superior mesenteric plexus) decreased average daily blood glucose measurements, compared to either single site treatment (i.e., liver or gastrointestinal stimulation alone) or sham controls;

FIG. 4 depicts HOMA-IR values illustrating that improved glucose control in the pFUS treated animals was not associated with an increased secretion of insulin, but rather restoration of a more insulin sensitive phenotype;

FIG. 5 depicts HOMA-B values illustrating that improved glucose control in the pFUS treated animals was not associated with an increased secretion of insulin, but rather restoration of a more insulin sensitive phenotype;

FIG. 6 depicts average insulin values to illustrate that improved glucose control in the pFUS treated animals was not associated with an increased secretion of insulin, but rather restoration of a more insulin sensitive phenotype;

FIG. 7 compares the glucose lowering effect of the dual-site pFUS to Metformin;

FIG. 8 compares the glucose lowering effect of the dual-site pFUS to Liraglutide;

FIG. 9 illustrates that while there was a transient or slowing effect on weight gain in the treated cohorts compared to sham controls, this effect was short-lived and did not explain the persistent improvement of hyperglycemia;

FIG. 10 illustrates that increasing the ultrasound treatment time resulted in an increase in the duration of hyperglycemia remission;

FIG. 11 illustrates the effects of the single pFUS treatment on diabetes remission and prevention;

FIG. 12 illustrates that remission lasted for the full 61-day experiment in the DIO animals;

FIG. 13 illustrates the effects of stimulation performed on cohorts with and without access to an exercise wheel;

FIG. 14 illustrates the effects of stimulation performed on cohorts with and without access to an exercise wheel and compared to sham controls with matched wheel access;

FIG. 15 illustrates observed values of pERK for sham and dual-site stimulation over time;

FIG. 16 illustrates observed values of pERK for sham, single-site, and dual-site stimulation;

FIG. 17 illustrates observed values of NPY for sham, single-site, and dual-site stimulation;

FIG. 18 illustrates observed values of POMC for sham, single-site, and dual-site stimulation;

FIG. 19 illustrates observed values of pERK for sham, single-site, and dual-site stimulation over time;

FIG. 20 illustrates observed values of NPY for sham, single-site, and dual-site stimulation over time;

FIG. 21 illustrates observed values of POMC for sham, single-site, and dual-site stimulation over time;

FIG. 22 illustrates observed values of NPY for sham and dual-site stimulation over time;

FIG. 23 illustrates observed values of POMC for sham and dual-site stimulation over time;

FIG. 24 demonstrates the effect of single versus dual-site pFUS treatment on circulating hormone concentrations;

FIG. 25 illustrates observed values of FGF1 for sham, single-site, and dual-site stimulation over time;

FIG. 26 illustrates observed values of FGF1 for sham, single-site, and dual-site stimulation;

FIG. 27 illustrates a schematic of a 3D in vitro peripheral neuron culture system and experimental setup used to capture both bright field and fluorescence images of DRG neuron cells before and after pFUS stimulation;

FIG. 28 illustrates zoom-in fluorescence images which show a time lapse of calcium (Ca2+) imaging during pFUS stimulation;

FIG. 29 illustrates that pFUS excitation of DRG neurons led to changes in Ca2+ dependent fluorescence (F);

FIG. 30 illustrates the observed fluorescence change before/after ultrasound stimulation using multiple ion channel blockers;

FIG. 31 illustrates that blocking TRPA1 abolished the ability of pFUS treatments to lower blood glucose levels;

FIG. 32 is a schematic representation of a neuromodulation system using a pulse generator according to embodiments of the disclosure; and

FIG. 33 is a block diagram of a neuromodulation system according to embodiments of the disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Any examples or illustrations given herein are not to be regarded in any way as restrictions on, limits to, or express definitions of, any term or terms with which they are utilized. Instead, these examples or illustrations are to be regarded as being described with respect to various particular embodiments and as illustrative only. Those of ordinary skill in the art will appreciate that any term or terms with which these examples or illustrations are utilized will encompass other embodiments that may or may not be given therewith or elsewhere in the specification and all such embodiments are intended to be included within the scope of that term or terms. Language designating such non-limiting examples and illustrations includes, but is not limited to: “for example,” “for instance,” “such as,” “e.g.,” “including,” “in certain embodiments”, “in some embodiments”, and “in one (an) embodiment.”

To facilitate review of the following discussion and examples, the following definitions are provided, though it should be provided that other definitions may be intrudes or added as needed throughout the discussion. A “tissue” as used herein pertain to multi-cellular organism and relates to an organized community or collection of cells that together carry out a specific function associated with the tissue. Correspondingly, the particular role of a tissue within a given organism depends on the type(s) of cells that are contained in the tissue. “Immediate early genes” (IEGs) may be understood to be genes that are activated transiently and rapidly, such as in response to various cellular stimuli. Such IEGs may be considered a standing response mechanism that may be activated (at the transcription level) as an initial or first-round of response to the given stimuli, such as before new proteins are synthesized. “Late response genes”, conversely, are only activated following the synthesis of early response gene products and, in the present context may be associated with long-term changes in neuronal function. In particular, in the present context late response genes may be associated with long term plasticity (e.g., long-term changes in synaptic function), requiring or associated with the synthesis of new mRNA and proteins. As further used herein, “ion channels” are specialized proteins found in a plasma membrane. Such ion channels function as a passageway through which charged ions cross through the plasma membrane down their electrochemical gradient.

Long-term remission from hyperglycemia has been demonstrated in type 2 diabetes (T2D) models using an invasive neuro-pharmaceutical approach in which a single treatment with recombinant fibroblast growth factor 1 (FGF1) has been observed to induce remission from hyperglycemia in diabetic mice and rats for weeks to months. However, this treatment involves application via invasive intracerebroventricular (i.e.v.) injection, into the third ventricle, which incurs the risk associated with such an invasive, intracranial procedure. Mechanistically, it is believed that this treatment modulates neurons within the arcuate nucleus of the hypothalamus. The basal hypothalamic nuclei are known to integrate signals from metabolic hormones, nutrients, and afferent neurons, and orchestrate the appropriate metabolic response to maintain energy homeostasis.

With the preceding in mind, the presently disclosed techniques demonstrate that long-term remission from hyperglycemia may be induced using physical neuromodulation (e.g., nerve stimulation) of afferent peripheral nerve pathways in communication with the hypothalamic metabolic nuclei. Certain implementations of such an approach are based on the use of focused, pulsed ultrasound stimuli to modulate the activity of peripheral nerve pathways (a technique referred to as peripheral focused ultrasound stimulation (pFUS)). Unlike electrical and implant-based nerve stimulation, ultrasound-based stimulation of the peripheral nerves is non-invasive and may be image-targeted, enabling precision stimulation of neurons or neuroendocrine cells associated with specific anatomical locations and functions.

Of note, as discussed herein, and with respect to the underlying mechanism of operation, in certain embodiments specific ultrasound parameters (e.g., length or duration of treatment, total delivered dose, and so forth) lead to activation of late response genes, namely the gene associated with FGF1 production, and to long-term therapeutic changes to the nerve pathway. This is believed to be the mechanism responsible for the long term diabetes remission observed in the diabetic animals models described herein. By way of further elaboration, and as discussed herein, stimulation of identified tissues (e.g., peripheral neural pathways) is performed for a sufficient length of time to induce a sustained change in membrane polarization in the plasma membrane or the nuclear membrane of certain cells. This change leads to sustained therapeutic changes, which in described examples is a change in the neural tissue of the hypothalamus. In certain implementations energy is directed to a target tissue (e.g., the hepatoportal plexus and/or superior mesenteric plexus) for a duration effective to activate late response genes (e.g., the FGF1 gene) in the hypothalamus. In particular, ion channel activation within the cells leads to sustained expression of immediate early genes, which leads to activation of the late response genes in question.

In an embodiment discussed herein the neuromodulation technique includes a neuromodulation treatment at two or more different regions of interest to target different parts of a physiological control pathway. In embodiments of these techniques, neuromodulation at a first site can be used to enhance or otherwise work in conjunction with neuromodulation at a second or different site or different sites in the patient. Thus, the enhancement may achieve unexpected results in metabolic pathway regulation.

As discussed herein, ultrasound can stimulate peripheral nerve fields (both efferent and afferent nerve fields) in and around organs. Provided herein, in an embodiment, are techniques to improve the anti-diabetic effects of ultrasound treatment that result in an unexpected outcome of providing long-term remission of type 2 diabetes, even in a genetic Fatty Diabetic Zucker (ZDF) Rodent model. In an embodiment, the techniques include a dual stimulation treatment of the hypothalamic metabolic control center using both ultrasound neuromodulation of a sensory field (i.e. hepatic/hepatoportal plexus neuromodulation of the ascending glucose sensor afferent pathway) and ultrasound neuromodulation of a neuroendocrine field (i.e. stimulation of glucagon-like peptide (GLP) secretion and associated hormonal and afferent pathway) from the gastrointestinal tract. In an embodiment the neuromodulation technique includes targeting the superior mesenteric plexus, the inferior mesenteric plexus, and/or the fundus of the stomach as a target for neuromodulating energy.

The disclosed parts of the neuromodulation techniques may be administered contemporaneously or may involve time-separated administration of different parts of the multi-site treatment (e.g., dual-site). In one embodiment, different target regions (i.e., dual-sites) may be neuromodulated within 30 minutes of one another or within 60 minutes of one another. In one embodiment, different target regions may be neuromodulated with at least a five minute separation. Provided herein are example time points between multi-site stimulation that provide time-separated neuromodulation of the liver vs. GI tract that enables a synergistic multi-site effect. Neuromodulation treatments may be aligned with glucose kinetics caused by the first energy application of the multi-site neuromodulation. The different parts of a multi-site neuromodulation may be part of a treatment administered together at a single patient visit.

It should be understood that the multi-site (e.g., dual-site) neuromodulation techniques may be part of a treatment regimen of a patient including multiple neuromodulations administered on different days, weeks, or months. The multi-site neuromodulations as disclosed herein may be modified or adjusted over the course of the treatment regimen based on patient progress or clinical condition. Adjustment may include adjusting energy application parameters or other relevant treatment parameters.

Unexpected effects of the multi-site neuromodulation techniques include long-term remission in diabetic animal models. Further unexpected effects include activation of late response genes in nerve pathways via ultrasound-based neuromodulation and the establishment of long-term remission in a disease (e.g., a metabolic disease) via therapeutic stimulation of nerve pathways. Such activation of late response genes may further implicate, prior to activation of the late response gene, sustained expression of immediate early genes for a sufficient duration (i.e., an effective duration) so as to activate the late response gene.

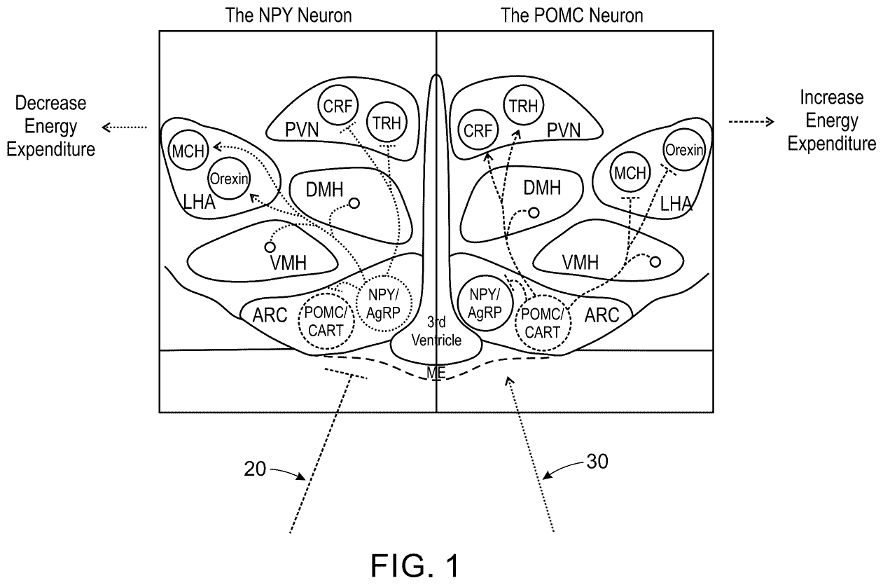

With this context in mind and as discussed herein, in certain embodiment the combined effect of peripheral focused ultrasound stimulation (pFUS) treatment at multiple neurometabolic sites (e.g., two separate peripheral sites stimulated serially (e.g., with between 1 minute to 60 minutes, 1 minute to 45 minutes, 1 minute to 30 minutes, 1 minutes to 15 minute, 1 minutes to 10 minute, or 30 seconds to 5 minutes between treatment at the different sites) during each treatment session) is employed. By way of further background and context, two nerve types modulating metabolic homeostasis have been identified: the neuropeptide Y (NPY) neurons and the proopiomelanocortin (POMC) neurons. Activation of the NPY neural path is associated with the orexigenic pathways and decrease energy expenditure at the metabolic level. Conversely, activation of the POMC neural path is associated with the anorectic pathways and increase energy expenditure at the metabolic level. The interplay between these two hypothalmic neuron types provide control over the metabolic system. As discussed herein, activation of the liver sensory pathway inhibits the NPY neuron (i.e., inhibits pathways that decrease energy expenditure in organs). Conversely, activation of the gut sensory pathway activates the POMC neuron (i.e., excites pathways that increase energy expenditure in organs). Thus, as shown in FIG. 1 and as discussed in greater detail herein, in accordance with certain embodiments, ultrasound liver stimulation 20 decreases NPY neuron activity while ultrasound gut stimulation 30 increases POMC neuron activity. With this context in mind, and as discussed in greater detail herein, in certain implementations precision ultrasound stimulation was performed on multiple ascending afferent pathways to the brain (and the neurons surrounded the 3rd ventricles) corresponding to the NPY neuron and POMC neuron as shown.

Further, as discussed herein, the FGF1 gene (i.e., the gene encoding FGF1) is a late response gene that is activated upon constitutive/long lasting stimulation of afferent neurons (that terminate in the metabolic control centrals within the arcuate nucleus and hypothalamus). As discussed herein, such activation of late response genes may involve prior to activation immediate early genes for a sufficient duration (i.e., an effective duration) so as to activate the late response gene. Thus, the neuromodulation described in the preceding passage is believed to correspondingly activate these late response genes (i.e., the FGF1 gene) to modulate the protein kinase R (PKR)-like endoplasmic reticulum kinase (pERK) pathway and thereby restore or improve insulin sensing and glucose signaling to this brain nuclei governing metabolic homeostasis. In particular, in neurons, FGF1 and late response gene activation is associated with activation of base-excision repair pathways, active DNA demethylation (i.e. epigenetic adaptations), and improvements in synaptic transmission and plasticity. Of particular note, while it has been recognized that these pathways may be “neuron activity dependent”, the present disclosure illustrates that these pathways can be activated with a therapeutic stimulus and that this activation depends on both the site(s) (anatomical site(s)) and duration of stimulation (e.g., activates after 30 minutes vs. 15 minutes or 5 minutes of stimulation). Thus, the present disclosure illustrates that there are non-invasive techniques that can “reactivate” dormant nerve pathways in diabetics, and re-establish healthy neuro-metabolic control.

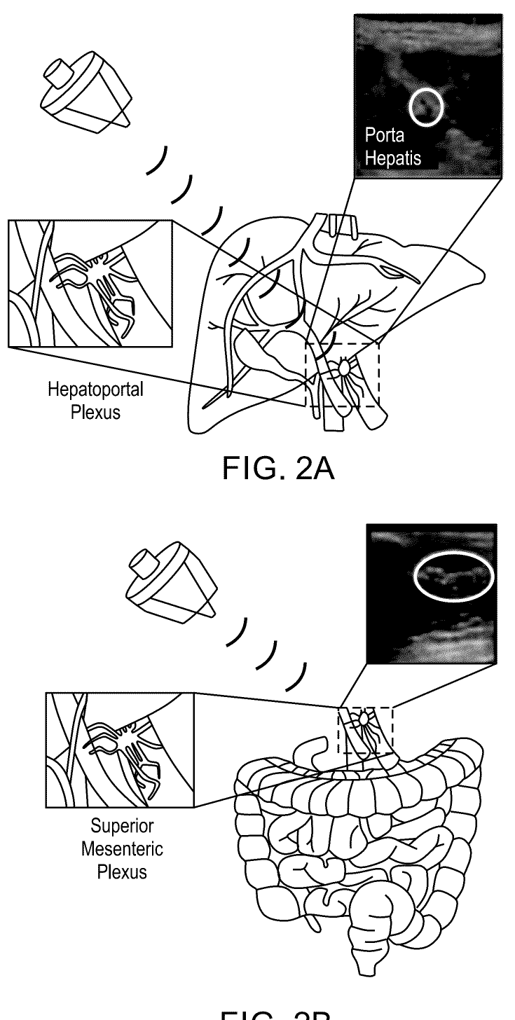

With the preceding in mind, FIGS. 2A and 2B illustrate the anatomical locations of the respective neuromodulation sites at the hepatoportal plexus (FIG. 2A: liver/hepatic site) and superior mesenteric plexus (FIG. 2B: gastrointestinal (GI) site) as discussed herein. Each of the liver and GI sites contain nutrient, hormone, and metabolite sensors and relay information on post-prandial nutrient status to the hypothalamus.

To perform the certain of the studies discussed herein, Zucker Diabetic Fatty (ZDF) animal models of T2D were selected for initial study. This selection was based on the previous intracerebroventricular injection (i.e.v.) FGF1 studies, which demonstrated months-long remission from hyperglycemia in several animal models, but remission of only ˜2 weeks in the ZDF model. In the ZDF model i.e.v. FGF1 also failed to elicit a sustained therapeutic effect in animals with severe hyperglycemia prior to treatment (i.e., blood glucose >300 mg/dL prior to treatment), possibly due to progressive deterioration of insulin sensitivity and development of severe insulin resistance in the ZDF model, which does not occur in other T2D models.

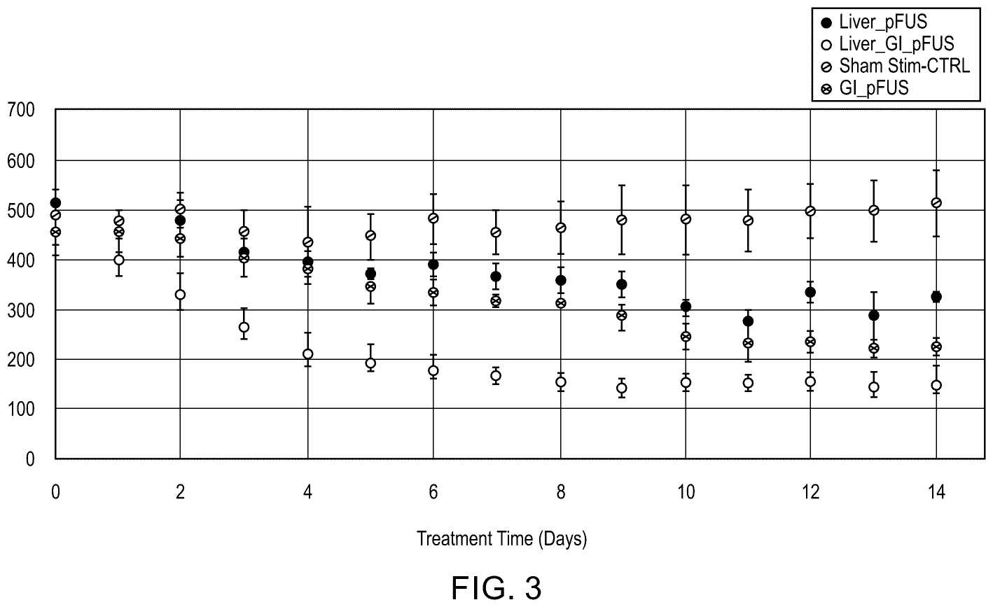

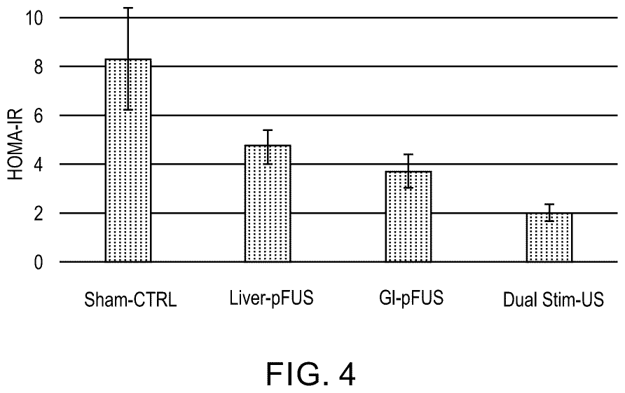

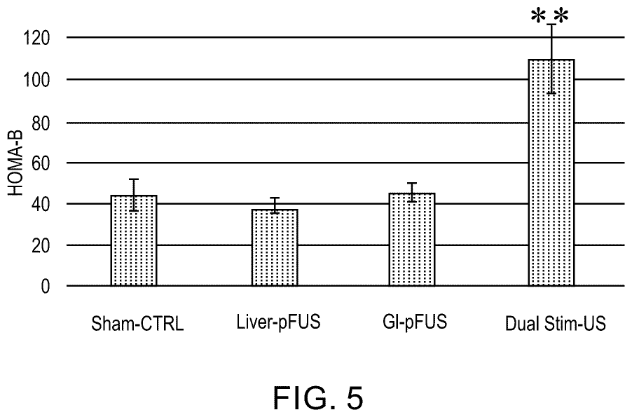

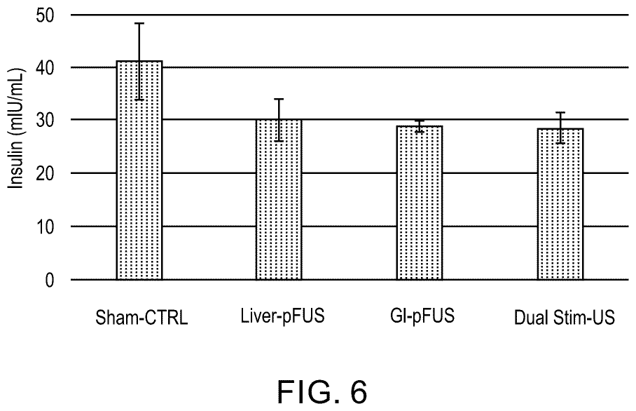

With respect to this study, FIG. 3 illustrates that dual-site pFUS of both the liver (i.e., hepatoportal plexus) and GI (i.e., superior mesenteric plexus) targets (shown as Liver_GI_pFUS in FIG. 3) decreased average daily blood glucose measurements in ZDF rats, compared to either single site treatment (i.e., liver or gastrointestinal stimulation alone) or sham controls. Blood glucose measures were started for all groups at age 50 days, and all animals demonstrated non-fasted glucose levels of 450-515 mg/dL prior to the start of the study. Daily dual-site pFUS (3-minute duration per day) resulted in a faster (i.e., average blood glucose values below 180 mg/dL by treatment day 7 compared to all other groups which remained above 300 mg/dL) and more significant (i.e., average blood glucose of 162.8+9.4 mg/dL versus 366.6+23.5 mg/dL in liver only and 319+26.7 mg/dL in GI only cohorts at day 14) reduction in average blood glucose values. Turning to FIGS. 4-6, HOMA-IR (FIG. 4), HOMA-B (FIG. 5), and average insulin values (FIG. 6) showed that the improved glucose control in the pFUS treated animals was not associated with an increased secretion of insulin, but rather restoration of a more insulin sensitive phenotype.

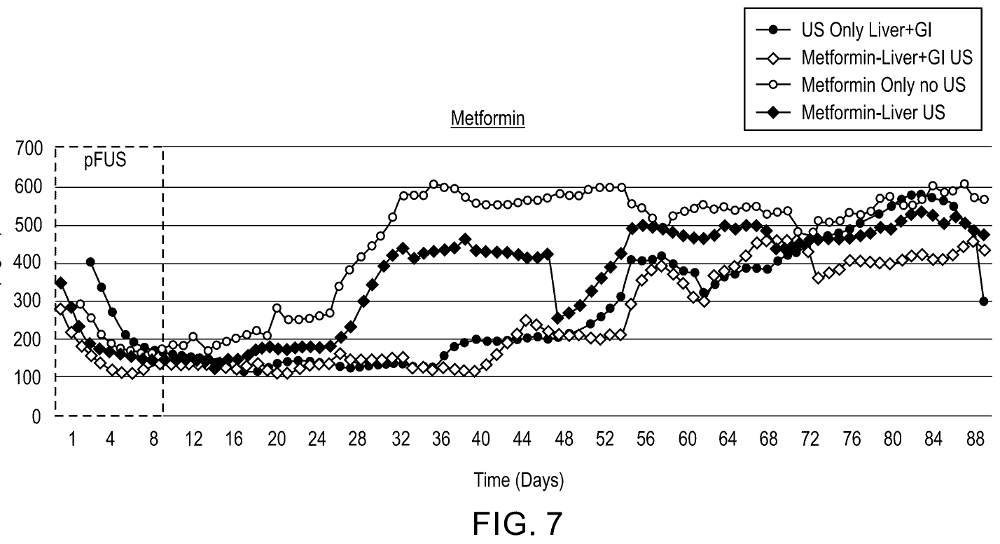

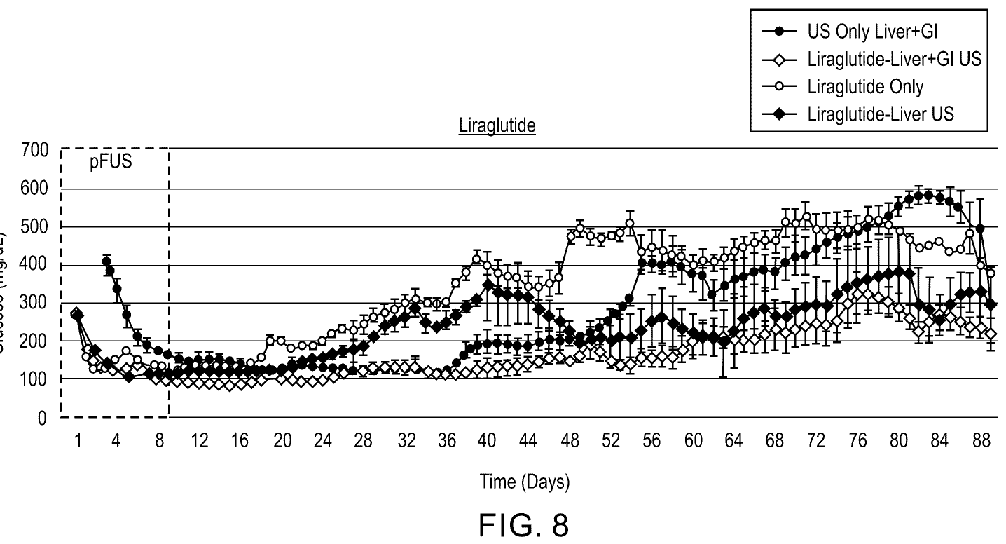

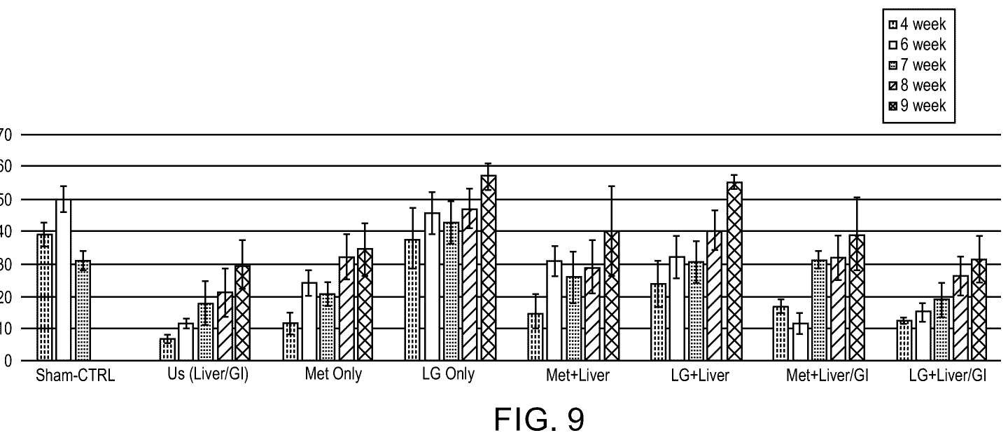

FIGS. 7 and 8 directly compare the glucose lowering effect of the dual-site pFUS to current T2D medications using the ZDF model. Drug only controls showed that with daily administration of metformin or liraglutide starting at day 50, average blood values in the ZDF rats return to hyperglycemic values (i.e., >200 mg/dL) within 19-20 days of daily drug treatment alone. In contrast, dual-site pFUS alone resulted in blood glucose values remaining below 200 mg/dL for 39 days. In addition, unlike the drug-treated cohorts (which continued to receive daily administration of the drug throughout the experiment), the dual-site pFUS cohorts in this experiment received only 10 days of daily (3-minute duration) ultrasound treatment and then no additional pFUS. In the combined treatment cohorts (i.e., 10-days of drug and pFUS treatment followed by daily treatment with drug alone), the addition of the initial ultrasound treatment resulted in an extended remission from hyperglycemia compared to the drug alone cohorts. Single-site, liver-only pFUS extended the effectiveness (as defined by time in which average blood glucose remains below 200 mg/dL) of the metformin treatment from 19 days to 27 days and the liraglutide treatment from 20 days to 30 days. The dual site pFUS treatment extended the effectiveness of the drug treatments even longer with the dual-site pFUS/metformin cohort remaining below 200 mg/dL for 44 days and the dual-site pFUS/liraglutide cohort achieving maintenance of sub-200 mg/dL glucose levels for 50 days (i.e., ZDF age 110 days). In addition, FIG. 9 shows that while there was a transient or slowing effect on weight gain in the treated cohorts compared to sham controls, this effect was short-lived and did not explain the persistent improvement of hyperglycemia.

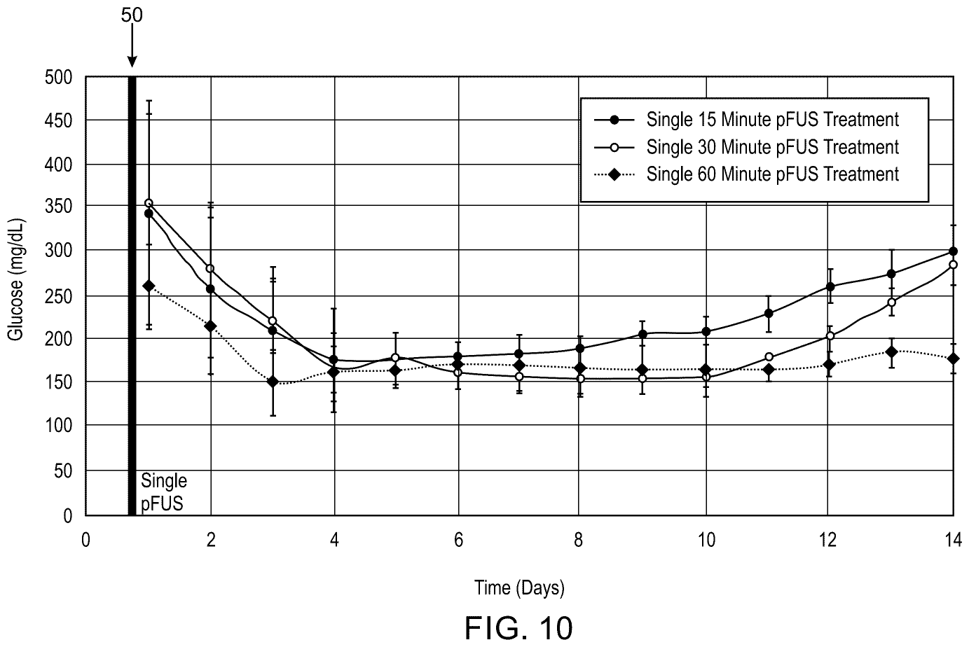

A further study was performed using a single dual-site pFUS treatment. In particular, the long-term effects were studied after only a single session of longer pFUS treatment durations of 15, 30, or 60 minutes (i.e., 7.5, 15, or 30-minutes treatments at each site in the same session). The ultrasound pulses used for pFUS remain below FDA safety limits of diagnostic ultrasound, and remained within an exposure time typically associated with clinical diagnostic imaging (i.e., 7.5 to 30 minutes per anatomical target). As shown in FIG. 10, increasing the ultrasound treatment time resulted in an increase in the duration of hyperglycemia remission in the ZDFs. The 15-minute treatment group maintained <200 mg/dL blood glucose for 8 days following the single dual-site pFUS treatment 50, while 30-minute treatments extended this remission time to 11 days and the 60-minute treatment resulted in remission over this initial full 14-day experiment.

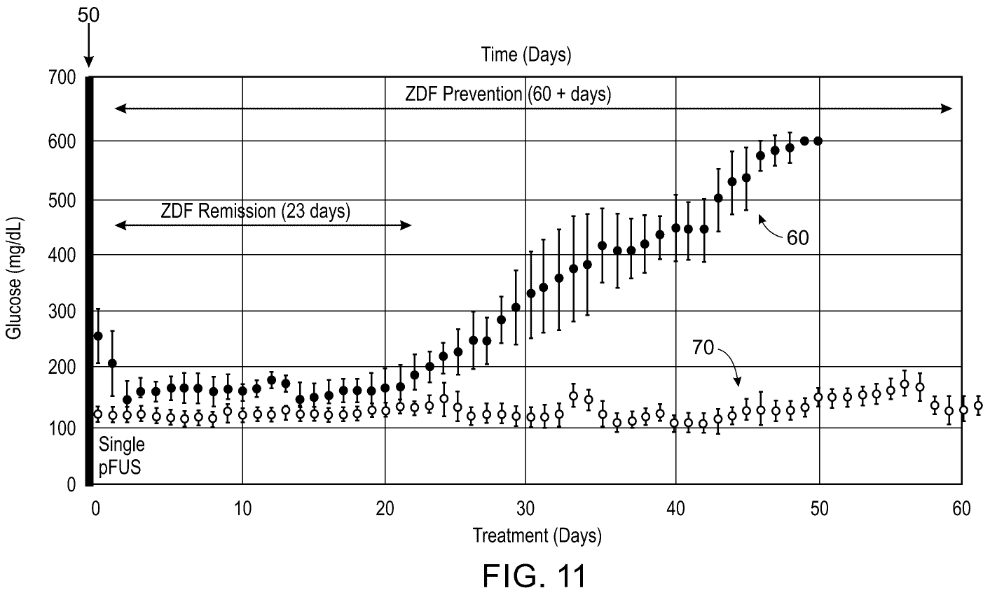

The single 60-minute dual-site pFUS treatment experiments were extended and tracked for over 60 days in both the ZDF and DIO rodent model. FIG. 11, shows the effects of the single pFUS treatment 50 on diabetes remission (i.e., pFUS treatment performed between days 55-60 of age, after hyperglycemia onset) and prevention (i.e., pFUS treatment performed at day 30 prior to hyperglycemia onset). In the previously diabetic ZDFs (line 60, treated at age 55 with starting glucose of 259.17+/−47 mg/dL), remission of glucose below 200 mg/dL lasted for 23 days, which is longer than the previously reported remission record in ZDFs (i.e., 19 days below 200 mg/dL from a single treatment of i.e.v. FGF1 injection). In the pre-diabetic ZDFs (line 70, treated at age 30 with starting glucose of 124.17+/−8.3 mg/dL (glucose intolerant pre-diabetic (indicated by fasting HOMA-IRs between 5.25+2.6 to 8.2+2.1 by 1-hour post glucose challenge, during oral glucose tolerance test (OGTT) of the onset of hyperglycemia/diabetes was prevented for the entire 61-day experiment.

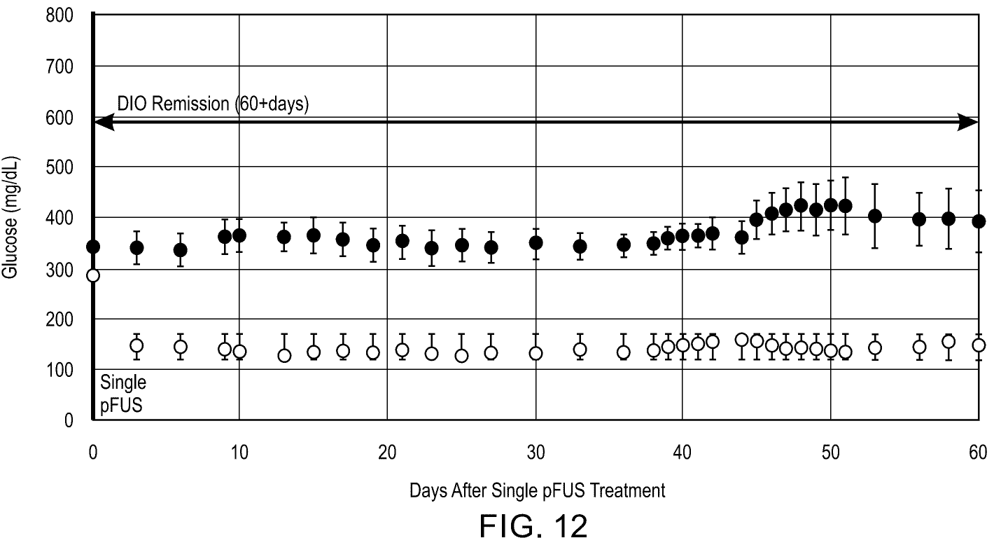

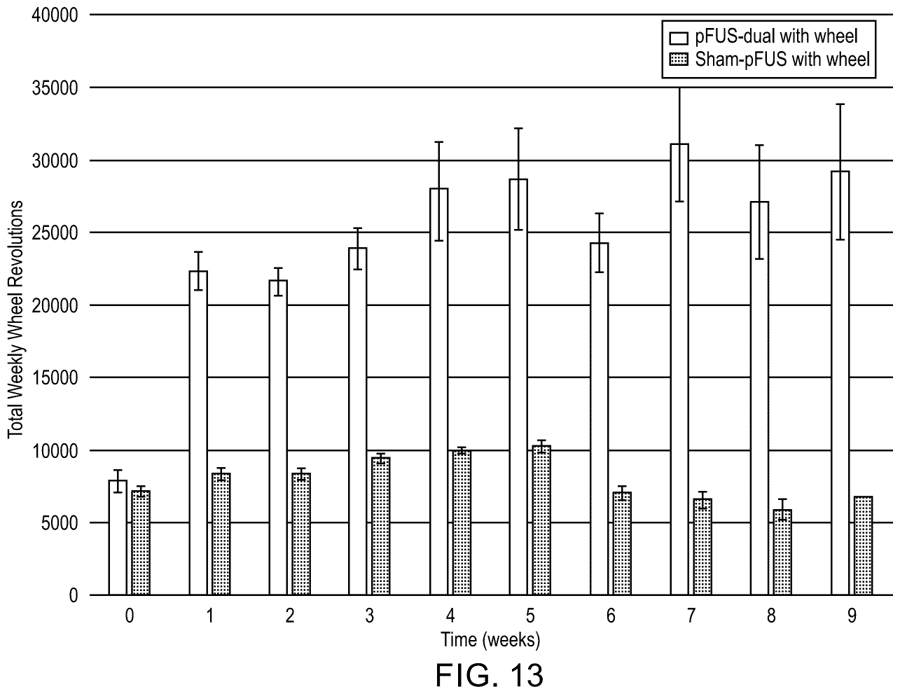

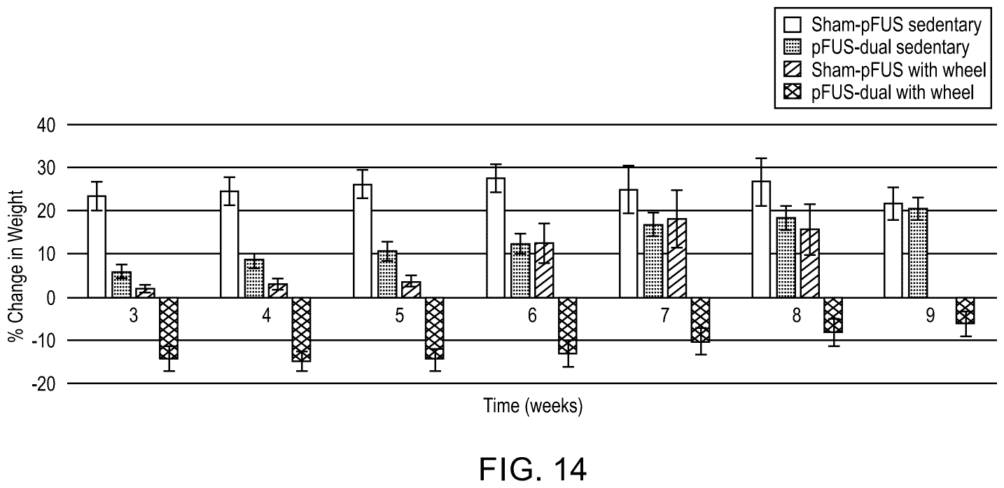

Remission also lasted for the full 61-day experiment in the DIO animals (FIG. 12; treated at age 55-60 with starting glucose of 285.33+/−43.9 mg/dL) following the single ultrasound treatment. Stimulation was also performed on DIO cohorts with and without access to an exercise wheel and compared to sham controls with matched wheel access (FIGS. 13 and 14). Like the ZDF cohorts, with no access to an exercise wheel, the ultrasound treatment slowed, but did not stop weight gain (8.5+/−4.3% versus 24.1+/−10.8% increase in the sham controls). However, when provided access to the exercise wheel the pFUS treated group lost 14.9+/−4.4% body weight (compared to a 2.9+/−2.1% weight gain in the exercised matched sham cohorts). This weight loss in the pFUS-treated animals (with wheel access) was associated with a significant increase in wheel usage (i.e., an average 27997 versus 9936 weekly wheel revolutions in pFUS treated versus sham control animals). This data further demonstrates that the glucose reduction provided by the ultrasound treatment is not secondary to weight loss (in cohorts without exercise wheel access) and may alter response to exercise (in cohorts with exercise wheel access).

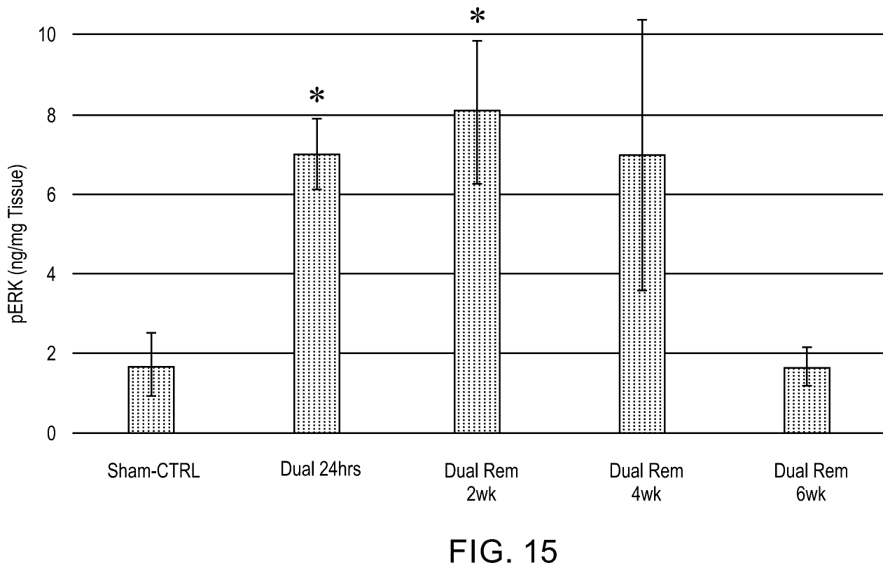

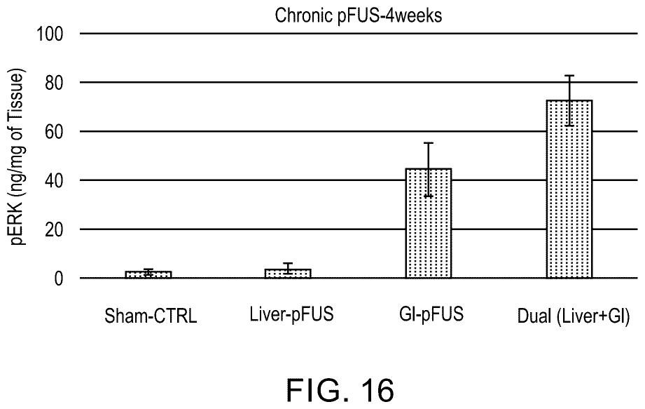

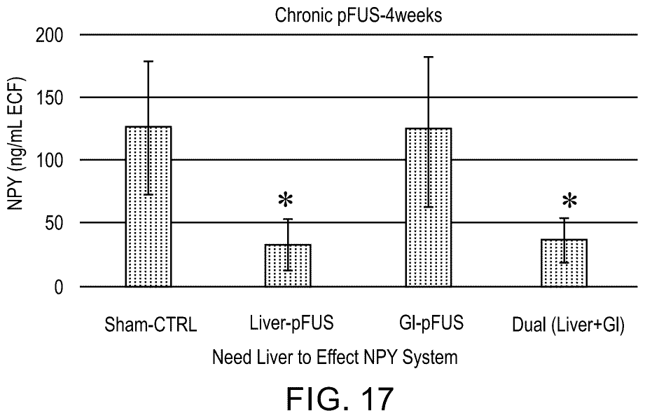

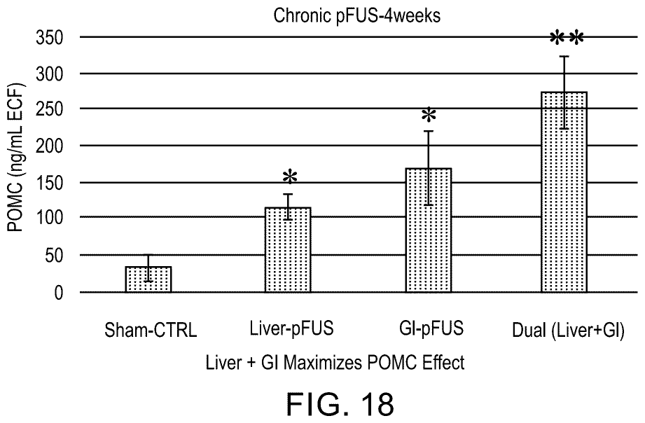

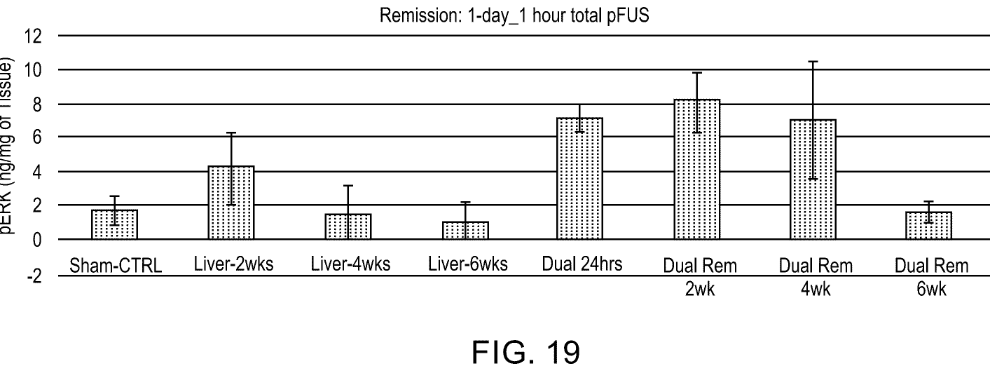

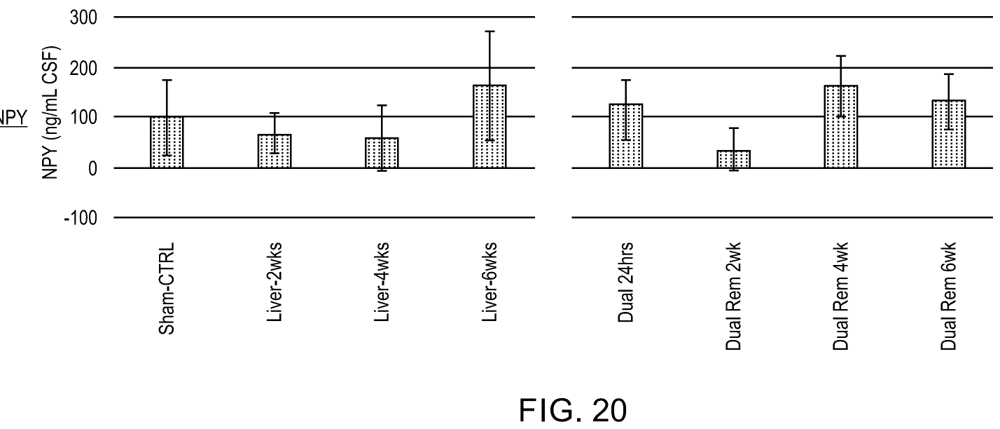

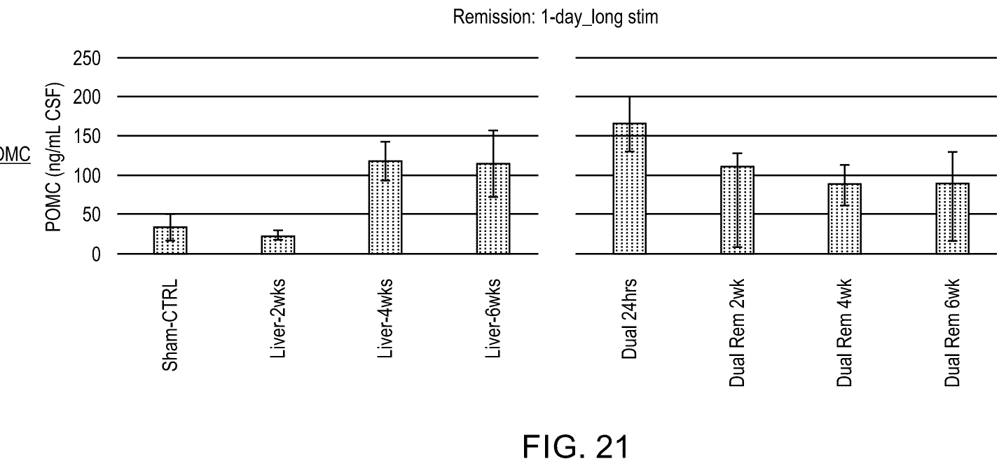

As noted herein, the capacity for FGF1 to induce diabetes remission when injected intracerebroventricularly was recently shown to be mediated by neurons located in the hypothalamic arcuate nucleus (ARC) and to require prolonged FGF-mediated induction of extracellular signal-regulated kinases ½ (ERK½). Therefore, the effect of dual-versus single-site pFUS on hypothalamic pERK concentrations was also examined. FIG. 15 shows that in the ZDF animals one dual-site pFUS treatment (which enabled >3-week remission from hyperglycemia as shown in FIG. 11) exerts a durable increase in hypothalamic ERK activity that persists for weeks. Furthermore, the onset of hyperglycemia (3-4 weeks after stimulation, as shown in FIG. 11) coincides with the return of hypothalamic pERK to pre-treatment levels (week 4; FIG. 15). This increase in hypothalamic ERK activity did not occur in the liver only pFUS cohorts (previously shown not to achieve diabetes remission in FIGS. 7 and 8) and was only observed to occur in GI stimulated cohorts (glucose values previously shown in FIG. 3) and occurred to a greater extent in dual-site treated animals compared to either liver—or GI-only stimulation (FIGS. 16-21).

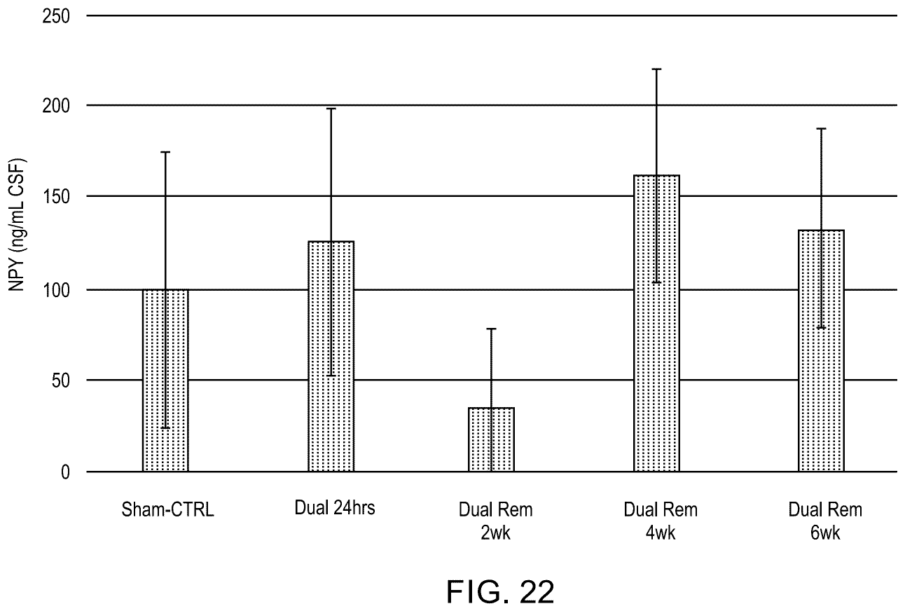

As previously noted, two distinct neuronal populations exist in the ARC that are known to coordinate energy homeostasis and regulate metabolism. These neural populations have opposite effects on feeding behavior and metabolism and include the anorexigenic pro-opiomelanocortin (POMC) neurons and the orexigenic neuropeptide Y (NPY)/agouti-related peptide (AgRP) neurons. Single-site liver pFUS treatment acts through liver-hypothalamic neural pathways to decrease hypothalamic NPY (and other neurotransmitters with known co-modulatory connections to NPY-expressing neurons). FIGS. 16-21 confirm this effect of liver only stimulation on hypothalamic NPY concentrations. Dual-site pFUS (FIG. 22) treatment significantly altered hypothalamic NPY levels. GI stimulation was shown to increase hypothalamic POMC levels (FIGS. 16-21), while dual-site treatment (versus GI site pFUS alone) resulted in an additional 2-fold increase in hypothalamic POMC (FIG. 23 and FIGS. 16-21). Furthermore, the dual-site pFUS treated animals (which demonstrated the 3-4 week remission from hyperglycemia in FIG. 15) sustained elevated hypothalamic POMC levels longer (up to 4 weeks following the single ultrasound treatment).

With the preceding in mind, it may be observed that neither NPY-only or POMC-only stimulation provide long term remission. As a result, it may be concluded that it is not just the length of time of stimulation, but that both pathways are activated for that length of time. Both pathways are connected by an integrating neuron, which may be an interneuron or cell in the central nervous system. By way of example, the pathways may be connected via an interneuron, non-neural supporting cells, and/or direct synaptic connections between the NPY neurons and POMC neurons. Because remission is only observed when both pathways are activated it is likely that the interneurons or other connections (e.g., non-neural supporting cells or direct synaptic connections) between the two neural pathways is relevant to the mechanism associated with remission. For example, it is possible that interneurons and/or direct synaptic connections between the NPY neurons and POMC neurons are involved in the late-response gene activation mechanism described herein in which both pathways are necessarily activated.

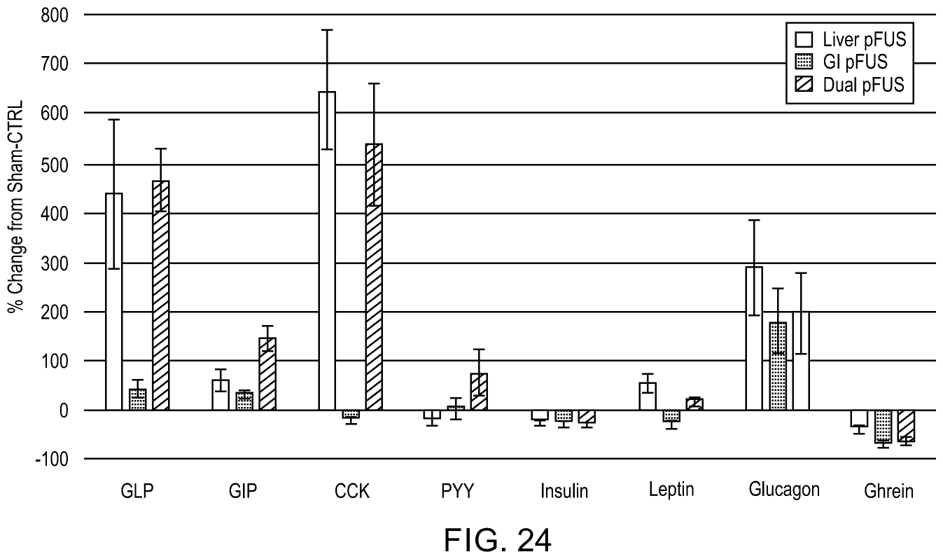

FIG. 24 demonstrates the effect of single versus dual-site pFUS treatment (i.e., daily stimulated ZDF cohorts from FIG. 3) on circulating hormone concentrations. Two of the five GI hormones (i.e., GLP and CCK) showed the largest percent change upon liver stimulation (either liver alone or in the dual stim cohort), whereas ghrelin was more effected by GI stim (either GI alone or in the dual stim cohort), GIP was effected by stimulation at both sites (but increased most significantly in the dual-stim cohort), and PYY was effected only in the dual-stimulation cohort. Thus, while both stimulation sites appear to effect concentrations of GI-related metabolic hormones, only the dual-site stimulation, likely mediated by the above-mentioned interneuron connection, provides an improvement across all measured hormones. Furthermore, while stimulation at either site (or in combination) resulted in similar improvements in circulating insulin and glucagon, only GI stimulation (either GI alone or in the dual-stim cohort) was effective at preventing an increase in circulating leptin levels. Hypothalamic effector pathways may effect secretion of several of these hormones and hormone secretion levels may help modulate hypothalamic nerve activity and alter glucose and energy homeostasis.

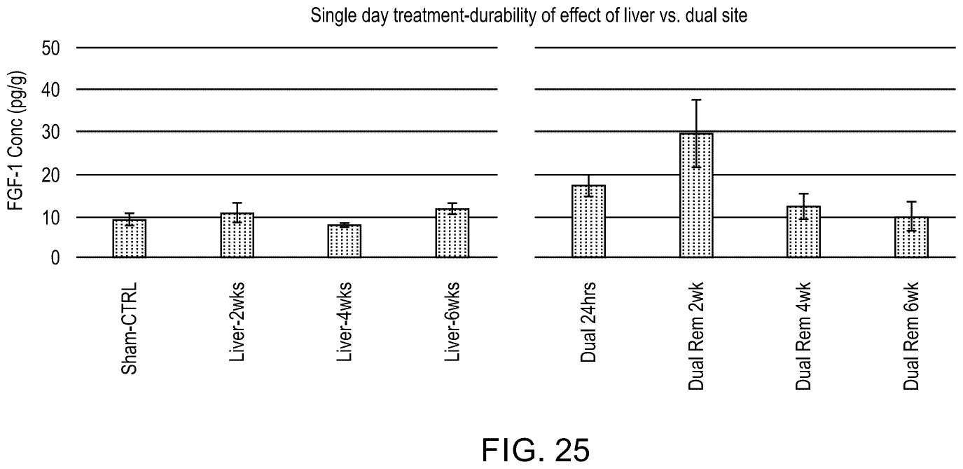

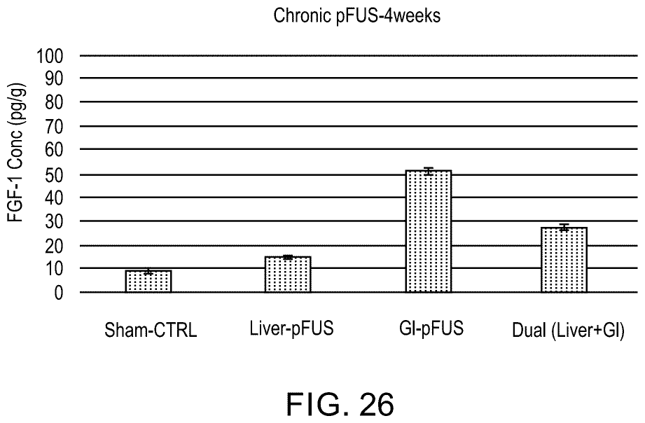

FGF1 concentration was measured following pFUS treatment to determine if prolonged pFUS modulation results in upregulation of hypothalamic FGF1. FIG. 25 shows that the single dual-site pFUS treatment resulted in FGF1 upregulation for at least two weeks following treatment. Furthermore, this effect was not seen in the single site stimulation cohorts (FIGS. 25 and 26). These results suggests that activation of peripheral metabolic sensory neurons may be important in activating or maintaining hypothalamic FGF1 signaling in vivo, and it's known neuroprotective/differentiation effect on neurometabolic controls circuits.

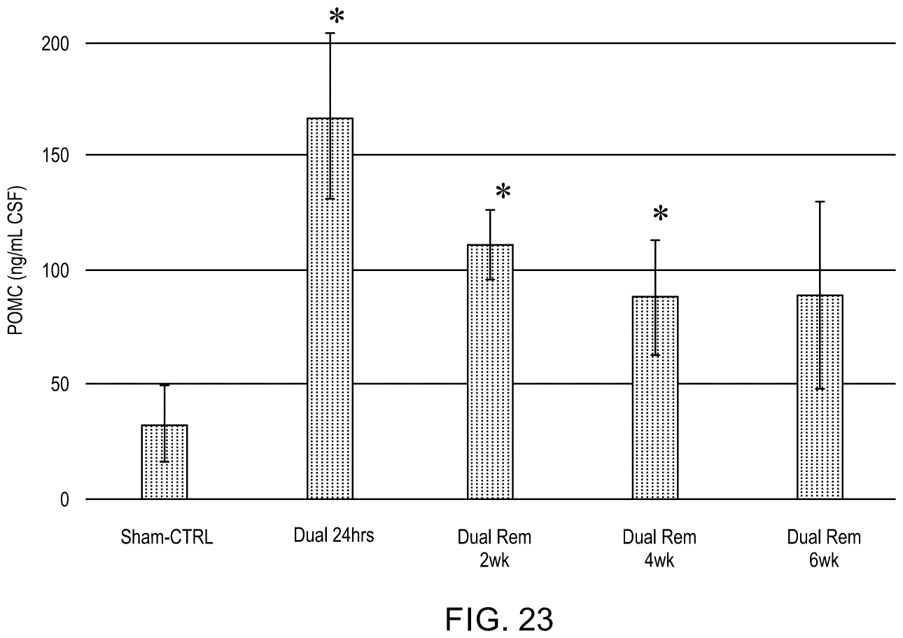

The hypothesis that intact afferent neural pathways between the metabolic sensory neurons (i.e., in liver and GI tract) and the hypothalamus were necessary to achieve pFUS-induced diabetes remission was also studied. Specifically, 0.5 μL of a 2% lidocaine hydrochloride solution was injected into the nucleus solitaris tractus (NTS) 60 minutes prior to the dual-site pFUS treatment. The lidocaine injection was observed to eliminate the dual-site-pFUS (60-minute duration) effect on glucose reduction, as measured by an oral glucose tolerance test (OGTT). Furthermore, in ZDF animals receiving the lidocaine block pFUS failed to induce an increase in pERK activity or FGF1 expression, compared to those animals without the block (FIGS. 15, 22, and 23) or receiving a saline-infection control. Similarly, the previously observed increase in POMC expression (FIG. 23) was absent in the ZDF animals receiving the nerve block prior to pFUS treatment.

Neurons in the hypothalamus (particularly in the ARC) are believed to play a key role in the maintenance of glucose homeostasis. Still, the ability of those hypothalamic nuclei, if modulated therapeutically, to ameliorate hyperglycemia in a sustained manner has only recently been shown in animal models. The only currently reported therapeutic mechanism of note to induce diabetes remission through hypothalamic neuromodulation has been i.e.v. (and not peripheral) injection of FGF1. The mechanism of action of i.e.v. FGF1 to induce diabetes remission involves modulation of glucoregulatory neurons within ARC through direct activation of FGF1-regulated intracellular signaling pathways (such as pERK). While the invasive nature of the central injection of FGF1 makes clinical testing and translation challenging, uncovering the mechanism of action provides further potential to investigate new therapeutic interventions. Herein, it has been disclosed that modulation of afferent nerve pathways communicating with the hypothalamus using ultrasound stimuli also resulted in sustained remission from hyperglycemia in multiple T2D models.

As discussed, FGF1 appears to be upregulated as a neural late response gene, whose expression levels and secretion rates depend on nerve activity and calcium concentration. Compared to early immediate early response genes, expression of late response genes (such as the FGF1 gene) requires constitutive nerve activation. As shown herein, induction of long-term remission in the diabetic model is ultrasound dose dependent (i.e., the length of remission depends on the length of stimulation time during treatment). A single treatment (e.g., a treatment of 15 minutes, 30 minutes, 60 minutes, and so forth) was shown capable of long-term amelioration of hyperglycemia in the ZDF and DIO model. This pFUS induced effect was shown to be dependent on intact neural pathways between the peripheral stimulation sites and the hypothalamus, and stimulation of different anatomical sites (i.e., the liver site known to contain glucose sensors or GI site known to contain nutrient sensors) was shown to have differing, but synergistic effects on hypothalamic neurotransmitter signaling. Hepatic pFUS primarily effected NPY expression within the hypothalamus while GI pFUS effected the levels of POMC, and dual stimulation of both sites was shown to achieve long-term remission. As with the i.e.v. FGF1 treatment, long-term remission after the dual-site pFUS was associated with effects on hypothalamic NPY, POMC, and pERK levels, and these hypothalamic effects coincided with pFUS-induced upregulation of FGF1 expression. With this in mind, modulation of neurons with the hypothalamus (i.e., either with neuropharmaceuticals or modulation with physical ultrasound stimuli) leads to sustained remission from diabetes in animal models. Furthermore, the ultrasound-based approach described herein is non-invasive (i.e., a single ultrasound treatment session using low intensity ultrasound stimulation).

With the preceding in mind, further discussion of the nature of the pFUS effect is discussed below to facilitate explanation. In particular, as described below the pFUS effect is dependent on the activity of mechanically activated ion channels, as may be observed in both in vivo and in vitro models.

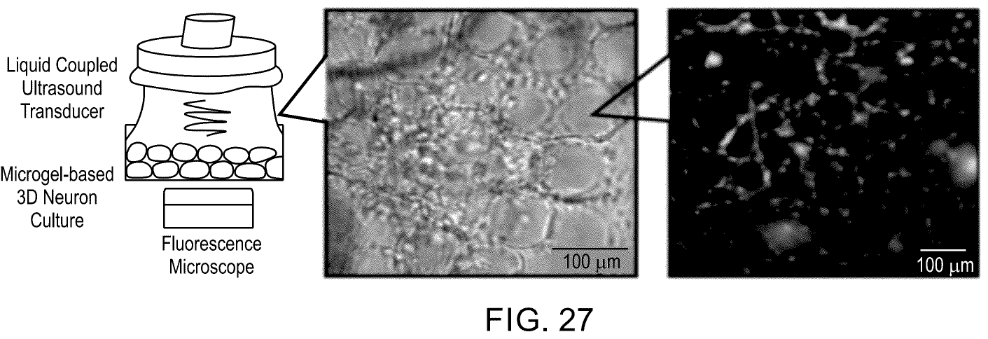

In particular, low intensity mechanical ultrasound stimuli are capable of site-specific nerve modulation in both in vivo and in vitro models. The mechanical origin of ultrasound neuromodulation is described here and was confirmed using a purely mechanical stimulus (i.e., replacing the ultrasound transducer with a mechanical piston-based stimulator and demonstrating the dependence of ultrasound nerve activation on specific families of mechano-sensitive ion channels, as shown in FIGS. 27-31. In the described study, three-dimensional in vitro cultures of dorsal root ganglia sensory neurons were activated (as measured by calcium indicator dye) using ultrasound pulse parameters and pressures that correspond to those from our in vivo experiments. With respect to the figures, FIG. 27 is a schematic of the 3D in vitro peripheral neuron culture system, and experimental setup used to capture both bright field and fluorescence images of DRG neuron cells before and after pFUS stimulation. The diameter of hydrogel particles ˜100 μm used for DRG neuron culture has previously been shown to create active axonal networks through the pores formed between hydrogel particles.

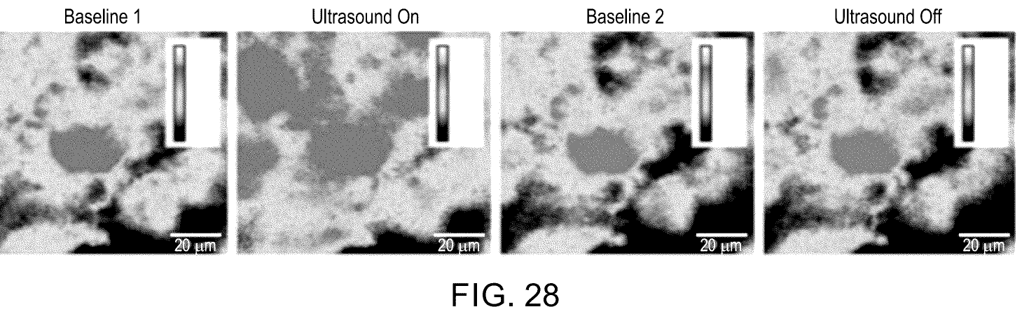

FIG. 28 illustrates zoom-in fluorescence images which show a time lapse of calcium (Ca2+) imaging during pFUS stimulation. pFUS stimulation is turned on at 10 s (ultrasound on) following a brief period of time for collecting baseline images (baseline 1) and turned off again at 120 s. Ultrasound was then turned off for 2 minutes prior to retaking a second baseline (baseline 2) and measurement period (ultrasound off) with the ultrasound stimulus turned off. Calcium concentrations of DRG neuron cells were increased after ultrasound stimulation, and upon pFUS cessation Ca2+ concentration in cells returned to baseline levels.

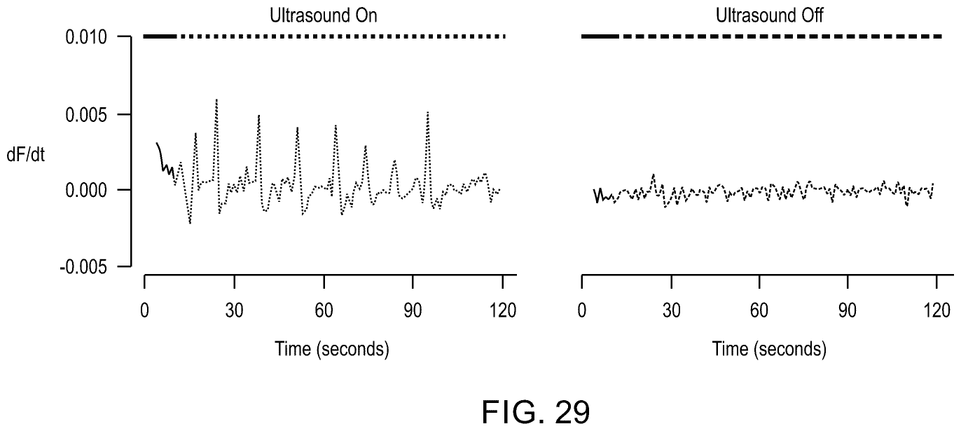

FIG. 29 illustrates that pFUS excitation of DRG neurons led to changes in Ca2+dependent fluorescence (F). The rate of change, dF/dt, remains small without application of ultrasound stimulus (ultrasound off). This is compared to the significant dF/dt increases shown during pFUS stimulation (ultrasound on) at 0.83 MPa peak-positive pressure) without any ion channel blocker added to the culture (N>30 cells/condition).

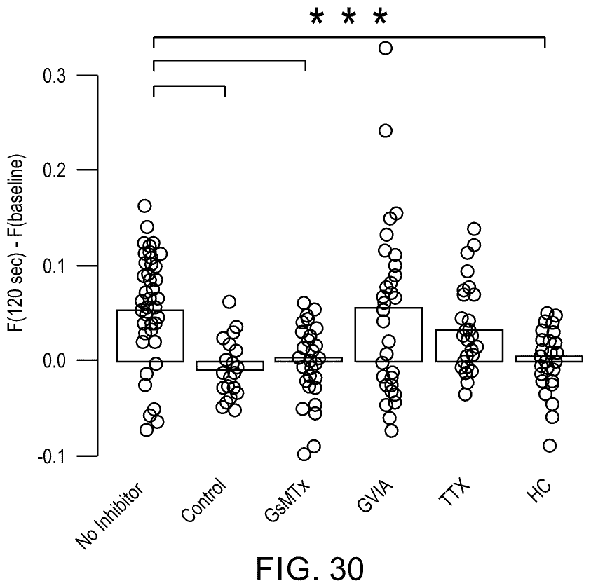

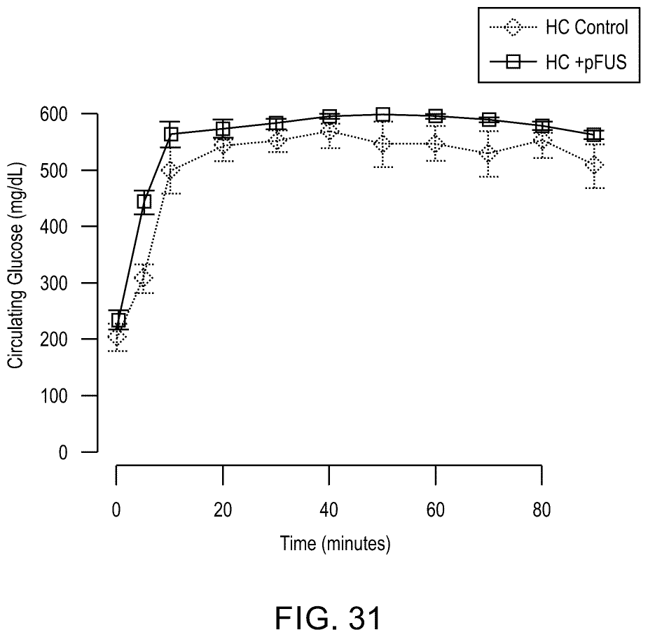

Turning to FIG. 30, observed fluorescence change before/after ultrasound stimulation using multiple ion channel blockers (N>30 cells/condition) is illustrated. TTX is a specific inhibitor of voltage-gated sodium channels involved in action potential propagation, w-conotoxin-GVIA is an N-type Ca2+channel inhibitor, and HC030031 (HC) is a sensitive transient receptor potential A1 (TRPA1) channel inhibitor. When piezo—or TRP-family blockers GsMTx and HC030031 were added to the culture, pFUS-induced activity was significantly suppressed (Dunnet's multiple test comparison; **** p<0.001). With respect to FIG. 31, HC030031 was shown to inhibit the pFUS effect on glucose in vivo when locally injected at the porta hepatis in ZDF rats (n=5 per group; GTT performed after overnight fast at age 65 days). The GTT was performed in both pFUS treated (HC+pFUS) and control animals given the HC injection but no pFUS (HC Sham control). Unlike ZDF rats without HC treatment, no statistical difference in circulating blood glucose was observed in animals treated with the TRPA1 blocker.

With further respect to these results, and by way of further explanation, blocking of N-type calcium channels (@-conotoxin) or voltage-gated sodium channels (tetrodotoxin) did not attenuate the response to pFUS, as shown in FIG. 30. In contrast, blockade using non-selective mechano-sensitive ion-channel blocker (i.e., GxMTx4) or specific blocker of transient receptor potential (TRPA1, i.e., HC-030031) channel inhibited the pFUS effect (FIG. 30). In order to determine if TRPA1 was also required to achieve the glucose lowering effect of hepatic pFUS in vivo, GTT studies were repeated after a single local injection of the TRPA1 blocker (HC-030031; 8 mg/kg) at the porta hepatis in a fasted ZDF rat. Confirming the important contribution of this channel, blocking TRPA1 indeed abolished the ability of pFUS treatments to lower blood glucose levels during GTT (FIG. 31). It may be noted that ion channels within the TRP family are expressed on afferent neurons and have been reported to be required for functional glucose/metabolite sensing. Furthermore, it was previously shown that activation of TRPA1 (target of HC-030031) by allyl isothiocyanate (AITC) improves glucose uptake and insulin signaling in multiple models of T2D by an unknown mechanism, while ablation of TRPV1 and TRPA1 expression (targets of GxMTx4) results in severe insulin or leptin resistance. From these results the interaction between calcium signaling and neuromodulation may be observed, as well as the dependence of the hypothalamic effect on TRPA1. It may also be noted that long-term calcium signaling to the nucleus is implicated in inducing late response gene activation, as described herein, and may thereby be a factor in late-response activation of the FGF1 gene. Thus the mechanism of FGF1 late-response gene activation may be related to TRPA1 activation.

### Neuromodulation Techniques

With the preceding in mind, neuromodulation of one or more regions of interest (e.g., single-site or multi-site) as provided herein permits a local and non-ablative application of energy to only the targeted region or regions of interest (e.g., to a stimulation site or sites) and without the energy being applied outside of the region or regions of interest. Energy application may trigger downstream effects outside of the targeted region of interest, e.g., in the same organ, tissue or structure containing the region of interest or in other organs and structures that do not contain the targeted region of interest. In some embodiments, the downstream effects may be induced in areas of a hypothalamus by way of example. The energy application may also induce effects along the targeted nerve upstream from the site of the energy application. In some embodiments, the effects outside of the targeted region/s of interest may be achieved without direct energy application to areas outside of the region/s of interest where the downstream effects or upstream effects are induced. Accordingly, local energy application may be used to realize or achieve systemic effects which may include local effects, downstream effects and/or upstream effects. The targeted region or regions of interest may be any tissue or structure in the body having axon terminals forming synapses with non-neuronal cells or fluids. In one example, the region of interest may be a subregion of an organ or structure, such as a spleen, liver, pancreas, or gastrointestinal tissue (i.e., “gut”). In another example, the regions of interest may be in a lymph system tissue.

Neuromodulation to the targeted regions of interest may exert a change in physiological processes to interrupt, decrease, or augment one or more physiological pathways in a subject to yield the desired physiological outcome. Further, because the local energy application may result in systemic changes, different physiological pathways may be changed in different ways and at different locations in the body to cause an overall characteristic profile of physiological change in the subject caused by and characteristic of the targeted neuromodulation for a particular subject. While these changes are complex, the present neuromodulation techniques provide one or more measurable targeted physiological outcomes that, for the treated subjects, are the result of the neuromodulation and that may not be achievable without the application of energy to the targeted region/s of interest or other intervention. Further, other types of intervention (e.g., drug treatment) may boost or enhance the physiological changes caused by neuromodulation.

The multi-site neuromodulation techniques discussed herein may be used to cause a physiological outcome of a change in concentration (e.g., increased, decreased) of a molecule of interest and/or a change in characteristics of a molecule of interest. That is, selective modulation of one or more molecules of interest (e.g., a first molecule of interest, a second molecule of interest, and so on) may refer to modulating or influencing a concentration (circulating, tissue) or characteristics (covalent modification) of a molecule as a result of energy application to one or more regions of interest (e.g., a first region of interest, a second region of interest, and so on) in one or more tissues (e.g., a first tissue, a second tissue, and so on). Modulation of a molecule of interest may include changes in characteristics of the molecule such as expression, secretion, translocation of proteins and direct activity changes based on ion channel effects either derived from the energy application itself or as a result of molecules directly effecting ion channels. Modulation of a molecule of interest may also refer to maintaining a desired concentration of the molecule, such that expected changes or fluctuations in concentration do not occur as a result of the neuromodulation. Modulation of a molecule of interest may refer to causing changes in molecule characteristics, such as enzyme-mediated covalent modification (changes in phosphorylation, aceylation, ribosylation, etc.). That is, it should be understood that selective modulation of a molecule of interest may refer to molecule concentration and/or molecule characteristics. The molecule of interest may be a biological molecule, such as one or more of carbohydrates (monosaccharaides, polysaccharides), lipids, nucleic acids (DNA, RNA), or proteins. In certain embodiments, the molecule of interest may be a signaling molecule such as a hormone (an amine hormone, a peptide hormone, or a steroid hormone).

| Certain embodiments described herein provide multi-site neuromodulation techniques that cause targeted physiological outcomes for the treatment of glucose metabolism and associated disorders. Glucose regulation is complex and involves different local and systemic metabolic pathways. Application of energy to targeted regions of interest causes characteristic changes in these metabolic pathways to improve glucose regulation. In some embodiments, modulation at one or more regions of interest may be used to treat disorders including but not limited to, diabetes (i.e., type 1 or type 2 diabetes), hyperglycemia, sepsis, trauma, infection, physiologic stress, diabetes-associated dementia, obesity, or other eating or metabolic disorders. In some embodiments, neuromodulation may be used to promote weight loss, control appetite, treat cachexia, or increase appetite. In one example, physiologic stress may be medically defined to include a variety of acute medical conditions (infection, severe injury/trauma, heart attack, bypass) as well as surgical instances with presentation of hyperglycemia. For example, direct pancreatic stimulation may result in increased appetite, while direct liver stimulation may cause a decrease in NPY, which in turn promotes signals of satiety. The targeted physiological outcome may include tuning circulating (i.e., blood) glucose concentrations in a subject to be within a desired concentration range associated with normal glucose levels and avoiding hyperglycemia or hypoglycemia. In this manner, selective modulation of a molecule of interest may be achieved. The tuning may be a result of induced changes in glucoregulatory hormones in the blood or tissue via targeted neuromodulation to cause the desired glucose concentration (i.e. desired glucose end point). Further, glucose regulation may be beneficial for healthy patients without a disease diagnosis, but who are pre-diabetic or who are hoping to maintain a healthy weight.

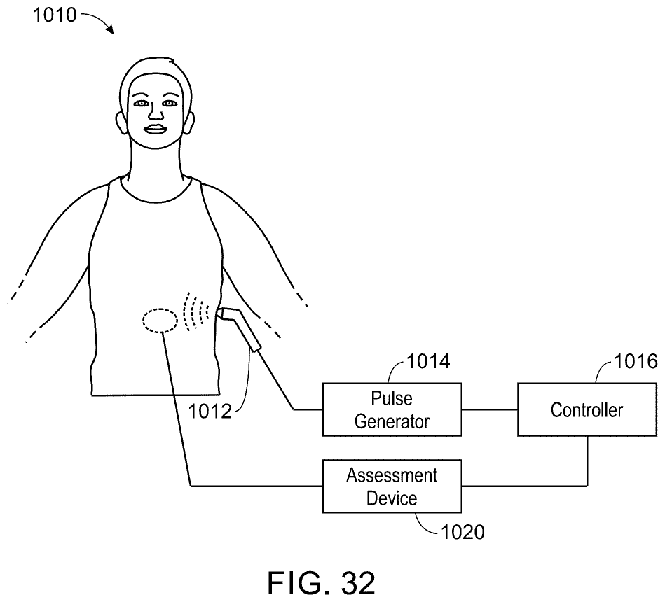

To that end, the disclosed neuromodulation techniques may be used in conjunction with a neuromodulation system. FIG. 32 is a schematic representation of a system 1000 for neuromodulation to achieve neurotransmitter release and/or activate components (e.g., the presynaptic cell, the postsynaptic cell) of a synapse in response to an application of energy. The depicted system includes a pulse generator 1014 coupled to an energy application device 1012 (e.g., an ultrasound transducer). The energy application device 1012 is configured to receive energy pulses, e.g., via leads or wireless connection, that in use are directed to a region of interest of an internal tissue or an organ of a subject, which in turn results in a targeted physiological outcome. In certain embodiments, the pulse generator 1014 and/or the energy application device 1012 may be implanted at a biocompatible site (e.g., the abdomen), and the lead or leads couple the energy application device 1012 and the pulse generator 1014 internally. For example, the energy application device 1012 may be a MEMS transducer, such as a capacitive micromachined ultrasound transducer.

In certain embodiments, the energy application device 1012 and/or the pulse generator 1014 may communicate wirelessly, for example with a controller 1016 that may in turn provide instructions to the pulse generator 1014. In other embodiments, the pulse generator 1014 may be an extracorporeal device, e.g., may operate to apply energy transdermally or in a noninvasive manner from a position outside of a subject's body, and may, in certain embodiments, be integrated within the controller 1016. In embodiments in which the pulse generator 1014 is extracorporeal, the energy application device 1012 may be operated by a caregiver and positioned at a spot on or above a subject's skin such that the energy pulses are delivered transdermally to a desired internal tissue. Once positioned to apply energy pulses to the desired site, the system 10 may initiate neuromodulation to achieve targeted physiological outcome or clinical effects.

In certain embodiments, the system 10 may include an assessment device 1020 that is coupled to the controller 1016 and assesses characteristics that are indicative of whether the targeted physiological outcome of the modulation have been achieved. In one embodiment, the targeted physiological outcome may be local. For example, the modulation may result in local tissue or function changes, such as tissue structure changes, local change of concentration of certain molecules, tissue displacement, increased fluid movement, etc.

The modulation may result in systemic or non-local changes, and the targeted physiological outcome may be related to a change in concentration of circulating molecules or a change in a characteristic of a tissue that does not include the region of interest to which energy was directly applied. In one example, the displacement may be a proxy measurement for a desired modulation, and displacement measurements below an expected displacement value may result in modification of modulation parameters until an expected displacement value is induced. Accordingly, the assessment device 1020 may be configured to assess concentration changes in some embodiments. In some embodiments, the assessment device 1020 may be an imaging device configured to assess changes in organ size and/or position. While the depicted elements of the system 10 are shown separately, it should be understood that some or all of the elements may be combined with one another. Further, some or all of the elements may communicate in a wired or wireless manner with one another.

Based on the assessment, the modulation parameters of the controller 1016 may be altered. For example, if a desired modulation is associated with a change in concentration (circulating concentration or tissue concentration of one or more molecules) within a defined time window (e.g., 5 minutes, 30 minutes after a procedure of energy application starts) or relative to a baseline at the start of a procedure, a change of the modulation parameters such as pulse frequency or other parameters may be desired, which in turn may be provided to the controller 1016, either by an operator or via an automatic feedback loop, for defining or adjusting the energy application parameters or modulation parameters of the pulse generator 1014.

The system 1000 as provided herein may provide energy pulses according to various modulation parameters. For example, the modulation parameters may include various stimulation time patterns, ranging from continuous to intermittent. With intermittent stimulation, energy is delivered for a period of time at a certain frequency during a signal-on time. The signal-on time is followed by a period of time with no energy delivery, referred to as signal-off time. The modulation parameters may also include frequency and duration of a stimulation application. The application frequency may be continuous or delivered at various time periods, for example, within a day or week. The treatment duration may last for various time periods, including, but not limited to, from a few minutes to several hours. In certain embodiments, treatment duration with a specified stimulation pattern may last for one hour, repeated at, e.g., 72 hour intervals. In certain embodiments, treatment may be delivered at a higher frequency, say every three hours, for shorter durations, for example, 30 minutes. The application of energy, in accordance with modulation parameters, such as the treatment duration and frequency, may be adjustably controlled to achieve a desired result.

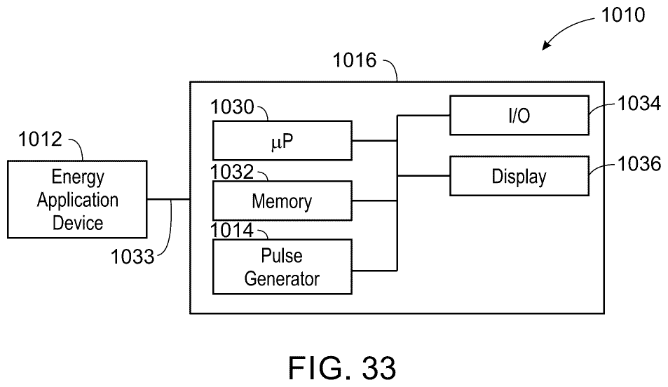

FIG. 33 is a block diagram of certain components of the system 1000. As provided herein, the system 1000 for neuromodulation may include a pulse generator 1014 that is adapted to generate a plurality of energy pulses for application to a tissue of a subject. The pulse generator 1014 may be separate or may be integrated into an external device, such as a controller 1016. The controller 1016 includes a processor 1030 for controlling the device. Software code or instructions are stored in memory 1032 of the controller 1016 for execution by the processor 1030 to control the various components of the device. The controller 1016 and/or the pulse generator 1014 may be connected to the energy application device 1012 via one or more leads 1033 or wirelessly

The controller 1016 also includes a user interface with input/output circuitry 1034 and a display 1036 that are adapted to allow a clinician to provide selection inputs or modulation parameters to modulation programs. Each modulation program may include one or more sets of modulation parameters including pulse amplitude, pulse width, pulse frequency, etc. The pulse generator 1014 modifies its internal parameters in response to the control signals from controller device 1016 to vary the stimulation characteristics of energy pulses transmitted through lead 1033 to an subject to which the energy application device 1012 is applied. Any suitable type of pulse generating circuitry may be employed, including but not limited to, constant current, constant voltage, multiple-independent current or voltage sources, etc. The energy applied is a function of the current amplitude and pulse width duration. The controller 1016 permits adjustably controlling the energy by changing the modulation parameters and/or initiating energy application at certain times or cancelling/suppressing energy application at certain times. In one embodiment, the adjustable control of the energy application device is based on information about a concentration of one or more molecules in the subject (e.g., a circulating molecule). If the information is from the assessment device 1020, a feedback loop may drive the adjustable control. For example, if a circulating glucose concentration, as measured by the assessment device 1020, is above a predetermined threshold or range, the controller 1016 may initiate energy application to regions of interest (e.g., liver and gastrointestinal tissue) and with modulation parameters that are associated with a reduction in circulating glucose. The initiation of energy application may be triggered by the glucose concentration drifting above a predetermined (e.g., desired) threshold or outside a predefined range. In another embodiment, the adjustable control may be in the form of altering modulation parameters when an initial application of energy does not result in an expected change in a targeted physiological outcome (e.g., concentration of a molecule of interest) within a predetermined time frame (e.g., 1 hour, 2 hours, 4 hours, 1 day).

In one embodiment, the memory 1032 stores different operating modes that are selectable by the operator. For example, the stored operating modes may include instructions for executing a set of modulation parameters associated with a particular treatment site, such as regions of interest in the liver, pancreas, gastrointestinal tract, spleen. Different sites may have different associated modulation parameters. Rather than having the operator manually input the modes, the controller 1016 may be configured to execute the appropriate instruction based on the selection. In another embodiment, the memory 1032 stores operating modes for different types of treatment. For example, activation may be associated with a different stimulating pressure or frequency range relative to those associated with depressing or blocking tissue function. In a specific example, when the energy application device is an ultrasound transducer, the time-averaged power (temporal average intensity) and peak positive pressure are in the range of 1 mW/cm²-30,000 mW/cm² (temporal average intensity) and 0.1 MPa to 7 MPa (peak pressure). In one example, the temporal average intensity is less than 35 W/cm² in the region of interest to avoid levels associated with thermal damage & ablation/cavitation. In another specific example, when the energy application device is a mechanical actuator, the amplitude of vibration is in the range of 0.1 to 10 mm. The selected frequencies may depend on the mode of energy application, e.g., ultrasound or mechanical actuator.

In another embodiment, the memory 1032 stores a calibration or setting mode that permits adjustment or modification of the modulation parameters to achieve a desired result. In one example, the stimulation starts at a lower energy parameter and increases incrementally, either automatically or upon receipt of an operator input. In this manner, the operator may achieve tuning of the induced effects as the modulation parameters are being changed.

The system 1000 may also include an imaging device that facilitates focusing the energy application device 1012. In one embodiment, the imaging device may be integrated with or the same device as the energy application device 1012 such that different ultrasound parameters (frequency, aperture, or energy) are applied for selecting (e.g., spatially selecting) a region of interest and for focusing energy to the selected region of interest for targeting and subsequently neuromodulation. In another embodiment, the memory 1032 stores one or more targeting or focusing modes that is used to spatially select the region of interest within an organ or tissue structure. Spatial selection may include selecting a subregion of an organ to identify a volume of the organ that corresponds to a region of interest. Spatial selection may rely on image data as provided herein. Based on the spatial selection, the energy application device 1012 may be focused on the selected volume corresponding to the region of interest. For example, the energy application device 1012 may be configured to first operate in the targeting mode to apply a targeting mode energy that is used to capture image data to be used for identifying the region of interest. The targeting mode energy is not at levels and/or applied with modulation parameters suitable for preferential activation. However, once the region of interest is identified, the controller 1016 may then operate in a treatment mode according to the modulation parameters associated with preferential activation.

The controller 1016 may also be configured to receive inputs related to the targeted physiological outcomes as an input to the selection of the modulation parameters. For example, when an imaging modality is used to assess a tissue characteristic, the controller 1016 may be configured to receive a calculated index or parameter of the characteristic. Based on whether the index or parameter is above or below a predefined threshold, the modulation parameters may be modified. In one embodiment, the parameter can be a measure of tissue displacement of the affected tissue or a measure of depth of the affected tissue. Other parameters may include assessing a concentration of one or more molecules of interest (e.g., assessing one or more of a change in concentration relative to a threshold or a baseline/control, a rate of change, determining whether concentration is within a desired range). Further, the energy application device 1012 (e.g., an ultrasound transducer) may operate under control of the controller 1016 to a) acquire image data of a tissue that may be used to spatially select a region of interest within the target tissue b) apply the modulating energy to the region of interest and c) acquire image to determine that the targeted physiological outcome has occurred (e.g., via displacement measurement). In such an embodiment, the imaging device, the assessment device 1020 and the energy application device 1012 may be the same device.

In another implementation, a desired modulation parameter set may also be stored by the controller 1016. In this manner, subject-specific parameters may be determined. Further, the effectiveness of such parameters may be assessed over time. If a particular set of parameters is less effective over time, the subject may be developing insensitivity to activated pathways. If the system 10 includes an assessment device 1020, the assessment device 1020 may provide feedback to the controller 1016. In certain embodiments, the feedback may be received from a user or an assessment device 1020 indicative of a characteristic of the target physiological outcome. The controller 1016 may be configured to cause the energy application device to apply the energy according to modulation parameters and to dynamically adjust the modulation parameters based on the feedback. For example, based on the feedback, the processor 1016 may automatically alter the modulation parameters (e.g., the frequency, amplitude, or pulse width of an ultrasound beam or mechanical vibration) in real time and responsive to feedback from the assessment device 1020.

In one example, the present techniques may be used to treat a subject with a metabolic disorder. The present techniques may also be used to regulate blood glucose level in subjects with disorders of glucose regulation. Accordingly, the present techniques may be used to promote homeostasis of a molecule of interest or to promote a desired circulating concentration or concentration range of one or more molecules of interest (e.g., glucose, insulin, glucagon, or a combination thereof). In one embodiment, the present techniques may be used to control circulating (i.e., blood) glucose levels. In one embodiment, the following thresholds may be used to maintain blood glucose levels in a dynamic equilibrium in the normal range:

### Fasted:

Less than 50 mg/dL (2.8 mmol/L): Insulin Shock
50-70 mg/dL (2.8-3.9 mmol/L): low blood sugar/hypoglycemia
70-110 mg/dL (3.9-6.1 mmol/L): normal
110-125 mg/dL (6.1-6.9 mmol/L): elevated/impaired (pre-diabetic)
125 (7 mmol/L): diabetic
   Non-Fasted (Postprandial Approximately 2 Hours after Meal):
70-140 mg/dL: Normal
140-199 mg/dL (8-11 mmol/L): Elevated or “borderline”/prediabetes
More than 200 mg/dL: (11 mmol/L): Diabetes

For example, the techniques may be used to maintain circulating glucose concentration to be under about 200 mg/dL and/or over about 70 mg/dL. The techniques may be used to maintain glucose in a range between about 4-8 mmol/L or about 70-150 mg/dL. The techniques may be used to maintain a normal blood glucose range for the subject (e.g., a patient), where the normal blood glucose range may be an individualized range based on the patient's individual factors such as weight, age, clinical history. Accordingly, the application of energy to one or more regions of interest may be adjusted in real time based on the desired end concentration of the molecule of interest and may be adjusted in a feedback loop based on input from an assessment device 1020. For example, if the assessment device 1020 is a circulating glucose monitor or a blood glucose monitor, the real-time glucose measurements may be used as input to the controller 16.

The energy application device 1012 may include an ultrasound transducer (e.g., a noninvasive or handheld ultrasound transducer) that is capable of applying energy to a target shown by way of non-limiting example as a liver. The energy application device 1012 may include control circuitry for controlling the ultrasound transducer. The control circuitry of the processor 1030 may be integral to the energy application device 1012 (e.g., via an integrated controller 1016) or may be a separate component. The ultrasound transducer may also be configured to acquire image data to assist with spatially selecting a desired or targeted region of interest and focusing the applied energy on the region of interest of the target tissue or structure based on the acquired image data.

The desired target within a region of interest may be an internal tissue or an organ that includes synapses of axon terminals and non-neuronal cells. The synapses may be stimulated by direct application of energy to the axon terminals within a field of focus of the ultrasound transducer focused on a region of interest of the target to cause release of molecules into the synaptic space. For example, the axon terminal forms a synapse with a liver cell, and the release of neurotransmitters and/or the change in ion channel activity in turn causes downstream effects such as activation of glucose metabolism. In one embodiment, liver stimulation or modulation may refer to a modulation of the region of interest at or adjacent to the porta hepatis. Similarly, gut or GI stimulation or modulation may refer to a modulation of the region of interest at or adjacent to the superior mesenteric plexus.

The energy may be focused or substantially concentrated on a region of interest and to only part of the internal tissue or organ, e.g., less than about 50%, 25%, 10%, or 5% of the total volume of the tissue. In one embodiment, energy may be applied to two or more regions of interest in the target tissue, and the total volume of the two or more regions of interest may be less than about 90%, 50%, 25%, 10%, or 5% of the total volume of the tissue. In one embodiment, the energy is applied to only about 1%-50% of the total volume of the tissue, to only about 1%-25% of the total volume of the tissue, to only about 1%-10% of the total volume of the tissue, or to only about 1%-5% of the total volume of the tissue. In certain embodiments, only axon terminals in the region of interest of the target tissue would directly receive the applied energy and release neurotransmitters while the unstimulated axon terminals outside of the region of interest do not receive substantial energy and, therefore, are not activated/stimulated in the same manner. In some embodiments, axon terminals in the portions of the tissue directly receiving the energy would induce an altered neurotransmitter release. In this manner, tissue subregions may be targeted for neuromodulation in a granular manner, e.g., one or more subregions may be selected. In some embodiments, the energy application parameters may be chosen to induce preferential activation of either neural or non-neuronal components within the tissue directly receiving energy to induce a desired combined physiological effect. In certain embodiments, the energy may be focused or concentrated within a volume of less than about 25 mm³. In certain embodiments, the energy may be focused or concentrated within a volume of about 0.5 mm³-50 mm³. A focal volume and a focal depth for focusing or concentrating the energy within the region of interest may be influenced by the size/configuration of the energy application device 1012. The focal volume of the energy application may be defined by the field of focus of the energy application device 1012.

As provided herein, the energy may be substantially applied only to the region or regions of interest to preferentially activate the synapse in a targeted manner to achieve targeted physiological outcomes and is not substantially applied in a general or a nonspecific manner across the entire tissue.

Technical effects of the disclosed embodiments include the use of therapeutic ultrasound to non-invasively stimulate multiple peripheral nerve pathways known to modulate energy homeostasis. An embodiment of the disclosed neuromodulation techniques includes neuromodulation techniques to treat a patient with a metabolic disorder. Certain embodiments of the disclosure are discussed in the context of blood glucose regulation.

This written description uses examples as part of the disclosure and also to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1-6. (canceled)

7. The system of claim 1, wherein the late-response gene comprises a fibroblast growth factor 1 (FGF1) gene.

8. The system of claim 1, wherein the late-response gene modulates a protein kinase R (PKR)-like endoplasmic reticulum kinase (pERK) pathway.

9-19. (canceled)

20. The system of claim 39, wherein the late-response gene causes production of FGF1.

21-29. (canceled)

30. A system for treating a metabolic disorder in a subject, the system comprising:
an energy application device comprising at least one ultrasound transducer configured to non-invasively target a tissue of the subject; and
a pulse generator configured to be connected to the at least one ultrasound transducer to stimulate the tissue using the at least one ultrasound transducer for a duration that is effective to thereby activate late response genes in the subject and thereby treat or alleviate the metabolic disorder.

31. The system of claim 30, wherein activating the late response genes comprises activating sustained expression of immediate early genes.

32. The system of claim 30, wherein the tissue comprises at least a portion of a peripheral nerve pathway.

33. The system of claim 32, wherein the peripheral nerve pathway comprises at least a portion of a hepatoportal plexus or a superior mesenteric plexus.

34. The system of claim 30, wherein the tissue comprises more than one peripheral nerve pathway, wherein the peripheral nerve pathways are in communication with each other or with an integrating neuron or cell in the central nervous system.

35. The system of claim 34, wherein the integrating neuron is an interneuron.

36. The system of claim 30, wherein the act of stimulating the tissue comprises activation of an ion channel selected from the TRP family of ion channels.

37. The system of claim 36, wherein the ion channel selected from the TRP family of ion channels comprises TRPA1.

38. The system of claim 30, wherein the act of stimulating the tissue comprises mechanical displacement of peripheral nerve pathways.

39. The system of claim 30, wherein the late response genes are in the hypothalamus of the subject.

40. The system of claim 39, wherein the late-response gene affects a concentration of glucose.

41. The system of claim 34, wherein the integrating neuron or cell connect neuropeptide Y (NPY) neurons and proopiomelanocortin (POMC) neurons.
